(19) United States
(12) Patent Application Publication
(10) Pub. No.: US 2025/0222120 A1
(43) Pub. Date: Jul. 10, 2025

(54) SYNTHESIS OF NOVEL SMALL MOLECULE CARM1 DEGRADERS, COMPOUNDS FORMED THEREBY, AND PHARMACEUTICAL COMPOSITIONS CONTAINING THEM

(71) Applicant: Wisconsin Alumni Research Foundation, Madison, WI (US)

(72) Inventors: Weiping Tang, Middleton, WI (US); Wei Xu, Middleton, WI (US); Haibo Xie, Madison, WI (US); Megan Bacabac, Madison, WI (US)

(73) Assignee: Wisconsin Alumni Research Foundation, Madison, WI (US)

(21) Appl. No.: 19/014,525

(22) Filed: Jan. 9, 2025

Related U.S. Application Data
(60) Provisional application No. 63/619,084, filed on Jan. 9, 2024.

(51) Int. Cl. A61K 47/64

(52) U.S. Cl. CPC A61K 47/64

(57) ABSTRACT

Provided herein are CARM1 PROTACs comprising a binding molecule of CARM1 with an optimized linker attached to an E3 ubiquitin ligase ligand that recruits the E3 ubiquitin ligase to CARM1. The PROTACs find use, e.g., for selectively targeted degradation of CARM1 protein in cancer cells. Also provided herein are compositions comprising the PROTACs, as well as methods of using the PROTACs.

### CROSS-REFERENCE TO RELATED APPLICATIONS

Priority is hereby claimed to provisional application Ser. No. 63/619,084, filed Jan. 9, 2024, which is incorporated herein by reference.

### FEDERAL FUNDING STATEMENT

This invention was made with government support under CA236356, CA268183 and CA281024 awarded by the National Institutes of Health. The government has certain rights in the invention.

### BACKGROUND

Methylation is one of the major post-translational modifications catalyzed by nine protein arginine methyltransferases (PRMTs) in mammalian cells (Yang and Bedford. “Protein Arginine Methyltransferases and Cancer” Nat. Rev. Cancer 2013, 13 (1), 37-50). PRMTs catalyze methyl group transfer from the S-adenosyl-L-methionine (SAM) to the two terminal nitrogen atoms of a protein arginine. They were classified into three types based on their enzymatic activities. The largest class Type I enzymes including PRMT1-4, 6 and 8, form asymmetric dimethylarginine (ADMA); Type II enzymes including PRMT 5 and 9 form symmetric dimethylarginine (SDMA); and Type III enzyme PRMT7 catalyzes mono-methylation on arginine. PRMT4, also known as coactivator-associated arginine methyltransferase 1 (CARM1), was first discovered as a nuclear hormone receptor coactivator through its association with p160 coactivators (Chen et al. “Regulation of Transcription by a Protein Methyltransferase” Science 1999, 284 (5423), 2174-2177). Through methylating histone H3R17 and H3R26 and numerous non-histone substrates, CARM1 is involved in various cellular processes (Suresh et al. “CARM1/PRMT4: Making Its Mark beyond Its Function as a Transcriptional Coactivator” Trends Cell Biol. 2021, 31 (5), 402-417).

CARM1 is amplified or overexpressed in many cancer types including breast cancer (Cheng et al. “Overexpression of CARM1 in Breast Cancer Is Correlated with Poorly Characterized Clinicopathologic Parameters and Molecular Subtypes” Diagn. Pathol. 2013, 8 (1), 129). Among breast cancer subtypes, CARM1 is overexpressed in triple negative breast cancer, which is typically associated with poor outcome (Davis et al. “Expression and Sub-Cellular Localization of an Epigenetic Regulator, Co-Activator Arginine Methyltransferase 1 (CARM1), Is Associated with Specific Breast Cancer Subtypes and Ethnicity” Mol. Cancer 2013, 12 (1), 40; Greenblatt et al. “CARM1 Is Essential for Myeloid Leukemogenesis but Dispensable for Normal Hematopoiesis” Cancer Cell 2018, 33 (6), 1111-1127). The oncogenic functions of CARM1 are strongly associated with its methylation of non-histone substrates. For example, methylation of BAF155, a subunit in the SWI/SNF complex, not only turns on massive oncogene expression, but also promotes tumor metastasis via an inhibiting antitumor immune response (Wang et al. “CARM1 Methylates Chromatin Remodeling Factor BAF155 to Enhance Tumor Progression and Metastasis” Cancer Cell 2014, 25 (1), 21-36). Overexpressing CARM1 in transgenic mouse models augmented mammary gland tumorigenesis in MMTV-Neu mice (Bao et al. “Mouse Models of Overexpression Reveal Distinct Oncogenic Roles for Different Type I Protein Arginine Methyltransferases” Cancer Res. 2019, 79 (1), 21-32). Using a genetically engineered CARM1 knockout (KO) cell line model, it was demonstrated that CARM1 KO significantly decreased the growth and invasion of breast cancer cells in vitro and in vivo, reinforcing that CARM1 is an important oncogene during breast cancer progression. (See Wang et al. “CARM1 Methylates Chromatin Remodeling Factor BAF155 to Enhance Tumor Progression and Metastasis” Cancer Cell 2014, 25 (1), 21-36; Shishkova et al. “Global Mapping of CARM1 Substrates Defines Enzyme Specificity and Substrate Recognition” Nat. Commun. 2017, 8 (1), 15571.)

Since 2008, potent small-molecule CARM1 inhibitors have been reported but none has moved to clinical investigation. While most of them are designed as substrate-competitive inhibitors, two of them are SAM competitors. (See Cai et al. “A Chemical Probe of CARM1 Alters Epigenetic Plasticity against Breast Cancer Cell Invasion” eLife 2019, 8, e47110; Iannelli et al. “Turning Nonselective Inhibitors of Type I Protein Arginine Methyltransferases into Potent and Selective Inhibitors of Protein Arginine Methyltransferase 4 through a Deconstruction-Reconstruction and Fragment-Growing Approach” J. Med. Chem. 2022, 65 (17), 11574-11606.) Although these inhibitors elicit low IC₅₀ in in vitro assays, high doses of the inhibitors are needed to inhibit the enzymatic activity of CARM1 in cell-based assays (Drew et al. “Identification of a CARM1 Inhibitor with Potent In Vitro and In Vivo Activity in Preclinical Models of Multiple Myeloma” Sci. Rep. 2017, 7 (1), 17993). Moreover, single-cell transcriptome analysis revealed the remarkable difference between inhibiting CARM1 and genetic knockout of CARM1 in cancer cells (Cai et al. “A Chemical Probe of CARM1 Alters Epigenetic Plasticity against Breast Cancer Cell Invasion” eLife 2019, 8, e47110). In breast cancer cells, CARM1 knockout, but not CARM1 inhibition, decreases cancer cell growth (Wang et al. “CARM1 Methylates Chromatin Remodeling Factor BAF155 to Enhance Tumor Progression and Metastasis” Cancer Cell 2014, 25 (1), 21-36; Kim et al. “BAF155 Methylation Drives Metastasis by Hijacking Super-Enhancers and Subverting Anti-Tumor Immunity” Nucleic Acids Res. 2021, 49 (21), 12211-12233). These studies suggest that CARM1 has non-enzymatic roles in driving cancer progression which necessitates the development of small molecule degraders of CARM1.

Proteolysis targeting chimera (PROTAC) is a transformative therapeutic paradigm to engage the body's natural protein disposal system for targeting disease-causing proteins with small molecules (Lai and Crews. “Induced Protein Degradation: An Emerging Drug Discovery Paradigm” Nat. Rev. Drug Discov. 2017, 16 (2), 101-114; Cromm and Crews. “Targeted Protein Degradation: From Chemical Biology to Drug Discovery” Cell Chem. Biol. 2017, 24 (9), 1181-1190; Békés et al. “PROTAC Targeted Protein Degraders: The Past Is Prologue” Nat. Rev. Drug Discov. 2022, 21 (3), 181-200). PROTACs consist of heterobifunctional small molecules bonded together via an appropriate linker (Sakamoto et al. “Protacs: Chimeric Molecules That Target Proteins to the Skp1-Cullin-F Box Complex for Ubiquitination and Degradation” Proc. Natl. Acad. Sci. U.S.A 2001, 98 (15), 8554-8559). The bifunctional nature in this case is defined by one end of the PROTAC binding with high selectivity to an E3 ubiquitin ligase while the other end simultaneously engages the target protein. As the binding event occurs, the exposed lysine residues of the target protein are brought in close proximity to the E3 ligase complex. As a result, the protein is poly-ubiquitinated and degraded by the proteasome. The PROTAC is released to continue its catalytic activity for targeted protein degradation. The recent discovery of potent small molecule E3 ligase ligands, including thalidomide analogs for cereblon (CRBN) and hydroxyproline derivatives for Von Hippel-Lindau (VHL), led to the surge of small-molecule PROTACs with high potency in cells since 2015. (See Winter et al. “Phthalimide Conjugation as a Strategy for in Vivo Target Protein Degradation” Science 2015, 348 (6241), 1376-1381; Bondeson et al. “Catalytic in Vivo Protein Knockdown by Small-Molecule PROTACs” Nat. Chem. Biol. 2015, 11 (8), 611-617; Lu et al. “Hijacking the E3 Ubiquitin Ligase Cereblon to Efficiently Target BRD4” Chem. Biol. 2015, 22 (6), 755-763; Zengerle et al. “Selective Small Molecule Induced Degradation of the BET Bromodomain Protein BRD4” ACS Chem. Biol. 2015, 10 (8), 1770-1777).) Degraders of disease-causing proteins have a number of potential advantages over inhibitors of oncogenic proteins. The occupancy-driven pharmacology of inhibitors requires stoichiometric drug binding to the oncogenic protein to inhibit the function of the protein. In contrast, induced protein degradation by PROTACs is event-driven and catalytic, providing favorable pharmacology (Ferreira de Freitas et al. “Discovery of a Potent and Selective Coactivator Associated Arginine Methyltransferase 1 (CARM1) Inhibitor by Virtual Screening” J. Med. Chem. 2016, 59 (14), 6838-6847). For example, it has been reported that a number of PROTAC degraders induce sustained tumor regression even after cessation of the PROTAC treatment (Burslem et al. “The Advantages of Targeted Protein Degradation Over Inhibition: An RTK Case Study” Cell Chem. Biol. 2018, 25 (1), 67-77.e3; Qin et al. “Discovery of QCA570 as an Exceptionally Potent and Efficacious Proteolysis Targeting Chimera (PROTAC) Degrader of the Bromodomain and Extra-Terminal (BET) Proteins Capable of Inducing Complete and Durable Tumor Regression” J. Med. Chem. 2018, 61 (15), 6685-6704; Li et al. “Discovery of MD-224 as a First-in-Class, Highly Potent, and Efficacious Proteolysis Targeting Chimera Murine Double Minute 2 Degrader Capable of Achieving Complete and Durable Tumor Regression” J. Med. Chem. 2019, 62 (2), 448-466; Burslem et al. “Targeting BCR-ABL1 in Chronic Myeloid Leukemia by PROTAC-Mediated Targeted Protein Degradation” Cancer Res. 2019, 79 (18), 4744-4753; Liao et al. “A PROTAC Peptide Induces Durable β-Catenin Degradation and Suppresses Wnt-Dependent Intestinal Cancer” Cell Discov. 2020, 6 (1), 1-12). Degraders based on inhibitors may still be effective for cancers that are resistant to the corresponding inhibitors in the case of target protein mutations. Mutations could reduce the binding affinity of inhibitors thus rendering drug resistance. Protein degraders are less sensitive to the decreased binding affinity as compared with the occupancy-driven inhibitors. (See Saenz et al. “Novel BET Protein Proteolysis-Targeting Chimera Exerts Superior Lethal Activity than Bromodomain Inhibitor (BETi) against Post-Myeloproliferative Neoplasm Secondary(s) AML Cells” Leukemia 2017, 31 (9), 1951-1961; Buhimschi et al. “Targeting the C481S Ibrutinib-Resistance Mutation in Bruton's Tyrosine Kinase Using PROTAC-Mediated Degradation” Biochemistry 2018, 57 (26), 3564-3575; Burslem et al. “Enhancing Antiproliferative Activity and Selectivity of a FLT-3 Inhibitor by Proteolysis Targeting Chimera Conversion” J. Am. Chem. Soc. 2018, 140 (48), 16428-16432; Zhang et al. “Protein Targeting Chimeric Molecules Specific for Bromodomain and Extra-Terminal Motif Family Proteins Are Active against Pre-Clinical Models of Multiple Myeloma” Leukemia 2018, 32 (10), 2224-2239.)

Recently, a PROTAC for Type II enzyme PRMT5 was developed as a chemical degrader for PRMT enzymes. (See Shen et al. “Discovery of First-in-Class Protein Arginine Methyltransferase 5 (PRMT5) Degraders” J. Med. Chem. 2020, 63 (17), 9977-9989.) The present disclosure seeks to address the unmet need for developing PROTACs targeting CARM1.

### SUMMARY

CARM1 is a protein that is amplified or overexpressed in many cancer types and its overexpression correlates with poor prognosis. Disclosed herein is a focused library of CARM1 protein degraders. The degraders use a binding molecule of CARM1 with an optimized linker attached to a ligand that recruits a ubiquitin ligase to CARM1. Upon ubiquitination, the cell removes CARM1 protein. The present disclosure describes the design, synthesis, and characterization of CARM1 PROTACs. It is shown that the CARM1 PROTACs developed herein promote rapid CARM1 degradation. The PROTACs do not induce other PRMT degradation and potently inhibit CARM1 substrate methylation in breast cancer cells. The PROTACs developed herein offer pharmacological advantages over inhibitors for the treatment of CARM1-driven cancers. The CARM1 degraders can also be used as chemical probes for studying the non-enzymatic functions of CARM1.

Thus, disclosed herein is a protein degrader, comprising a binding molecule configured to bind CARM1, a linker; and a ligand configured to bind an E3 ubiquitin ligase, wherein the binding molecule and the ligand are operationally linked by the linker.

In certain versions, the binding molecule of CARM1 is a small molecule. A non-limiting example of the binding molecule is TP-064.

The E3 ubiquitin ligase ligand may be a ligand of Von Hippel-Lindau (VHL) or cereblon (CRBN). In certain versions, the ligand is a VHL ligand.

The linker may comprise stable and flexible alkyl linkages, or the linker may be a more rigid linker comprising heterocycles. In certain versions, the linker is selected from:

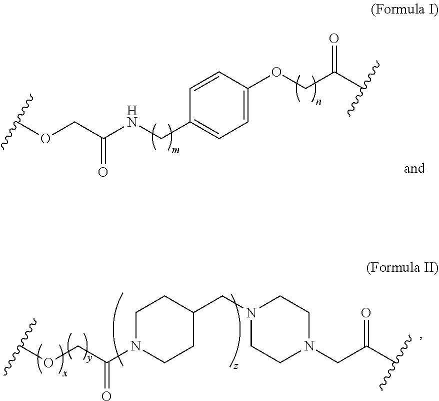

wherein m is 1 or 2; n is an integer from 3 to 7; x is 0 or 1; y is 0, 1, or 2; and z is 1 or 2.

A non-limiting example of the linker has the following structure:

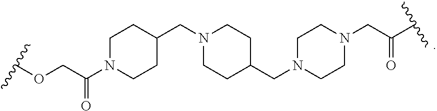 [SMILES: *OCC(=O)N1CCC(CN2CCC(CN3CCN(CC(*)=O)CC3)CC2)CC1]

Also provided herein is a pharmaceutical composition comprising the protein degrader of the present disclosure.

Also provided herein is a method of degrading CARM1 protein, comprising contacting the CARM1 protein with the protein degrader of the present disclosure.

Also provided herein is a method comprising administering to an individual in need thereof a therapeutically effective amount of the pharmaceutical composition of the present disclosure.

The objects and advantages of the disclosure will appear more fully from the following detailed description of the preferred embodiment of the disclosure made in conjunction with the accompanying drawings.

### BRIEF DESCRIPTION OF THE DRAWINGS

FIGS. 1A-1B. Quick identification of the linker length and E3 ubiquitin ligase for CARM1 PROTAC. (FIG. 1A) Crystal structure of CARM1 in complex with TP-064 (PDB: 5U4X). The highlighted phenyl ring of TP-064 is solvent-exposed. (FIG. 1B) The initial PROTAC library was derived from the coupling of the CARM1 ligand with a hydrazide functional group and a partial PROTAC library with a benzaldehyde functional group.

FIGS. 2A-2B. Rapid-TAC VHL based PROTACs induced degradation of CARM1 in MCF7 cells. (FIG. 2A) MCF7 cells were treated with the PROTACS at single concentrations (10 μM). (FIG. 2B) MCF7 cells were treated with TPVC3 at 0.01, 0.1, 0.5, 1, and 10 μM. Cell lysates were collected and the CARM1 levels were detected by western blots.

FIGS. 3A-3D. Structural activity relationship of CARM1 degraders. (FIG. 3A) Chemical structures of CARM1 degraders, 2a-2e bearing a flexible alkyl linker, 3a-3e bearing a rigid linker that contains the combination of piperidine and piperazine rings. (FIG. 3B) and (FIG. 3C) MCF7 cells were treated with the compounds at the indicated concentrations for 24 h. Cell lysates were collected and the CARM1 levels were detected by western blots. The results are representative of three independent experiments. (FIG. 3D) MCF7 cells were treated with DMSO and 0.5 μM of 3b, 3c, 3d, and 3e for 24 h.

FIGS. 4A-4F. Degradation activities of compounds 3b and 3e. (FIG. 4A) Chemical structure of 3b, 3e and 3bN. (FIG. 4B) MCF7 cells were treated with the compounds at 0.5 μM for indicated time. (FIG. 4C) and (FIG. 4D) MCF7 cells were treated with the compounds at the indicated concentrations for 24 h. Cell lysates were collected and the CARM1 levels were detected by western blots. The results are representative of three independent experiments. (FIG. 4E) and (FIG. 4F) DC₅₀ and Dₘₐₓ were plotted and calculated based on three independent biological triplicate western blot assays.

FIG. 5. CARM1 degradation activity of compound 3b in MCF10A cells. The MCF10A cells were treated with 3b at the indicated concentrations. Cell lysates were collected and the CARM1 levels were detected by western blots.

FIGS. 6A-6E. Mechanism and selectivity of degrader 3b. (FIG. 6A) MCF7 cells were co-treated with 0.5 μM of 3b and DMSO, 7.5 μM of MG132, 20 μM of VH-032, 2 μM of MLN4924, and 10 μM of TP-064 for 24 h. Cell lysates were collected and CARM1 levels were detected by Western blot. (FIG. 6B) MCF7 cells were treated with DMSO, 3b, or 3bN. Cell lysates were collected and CARM1 levels detected by Western blot. (FIG. 6C and FIG. 6D) MCF7 cells were treated with the indicated concentration of 3b for 24 h. Cell lysates were collected and PRMT1, PRMT6 and PRMT5 levels were detected by Western blot. (FIG. 6E) Volcano plots showed protein expression level changes for 3b versus DMSO group. Log 2 protein fold changes are plotted against the negative log 10 p-values. Proteins exhibiting significant alternations (p-value <0.05, Student's t-test) are represented by points above the non-axial horizontal line. Significantly down-regulated proteins are depicted in blue, while up-regulated proteins are depicted in red (protein |fold change|>2).

FIGS. 7A-7F. Biological effect comparison of inhibitor and degrader 3b in cell lines. (FIG. 7A) MCF7 cells were treated with indicated concentrations of TP-064 or 3b for 24 h. Cell lysates were collected and CARM1, methyl-BAF155, total BAF155, methyl-PABP1, and total PABP1 levels were detected by Western blot. (FIG. 7B) BT474 and (FIG. 7C) MDA-MB-231 cells were treated with DMSO, 10 μM of TP-064, and 0.5 μM of 3b for the indicated times. Cell lysates were collected and CARM1, methyl-BAF155, total BAF155 levels were detected by Western blot. (FIG. 7D) MDA-MB-231 cells were treated with DMSO, 0.5 μM of 3b, or 10 μM of TP-064 for 7 days. Cell proliferation rate normalized to day 0 was plotted. (FIG. 7E) and (FIG. 7F) Transwell cell migration assays after MDA-MB-231 cells were treated with DMSO, 0.5 μM 3b, or 10 μM TP-064. Migrated cells were stained with 1% crystal violet and the percentage of migrated cells were plotted. Data mean±s.d. ***. P≤0.001.

FIG. 8. Compound 3b cell proliferation assay in MCF7 and MCF10A cells. The MCF7 (upper panel) and MCF10A (lower panel) cells were treated with 3b at 0.5 μM.

FIGS. 9A-9B. Compound 3b colony formation assay in MDA-MB-231 cells. The MDA-MB-231 cells were treated with DMSO, 10 μM TP-064, 0.5 μM 3b and 3bN for two weeks. (FIG. 9A) Staining of the cells after treatment. (FIG. 9B) quantification of the intensity of the staining.

### DETAILED DESCRIPTION

### Abbreviations and Definitions

ADMA=Asymmetric dimethylarginine; BAF155=BRG1-associated factor 155;
CARM1=Coactivator-associated Arginine Methyltransferase 1;
CRBN=Cereblon; PABP1=Polyadenylate-binding protein 1;
PRMT=Protein arginine methyltransferases; PROTAC=Proteolysis targeting chimera;
SAM=S-adenosyl-L-methionine; SAR=Structure activity relationship;
SDMA=Symmetric dimethylarginine; TLC=Thin-layer chromatography;
TPD=Target protein degradation; VHL=Von Hippel-Lindau.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present disclosure shall include the corresponding plural characteristic or limitation, and vice-versa, unless otherwise specified or clearly implied to the contrary by the context in which the reference is made.

As used herein, the term “or” is an inclusive “or” operator and is equivalent to the term “and/or” unless the context clearly dictates otherwise.

The elements and method steps described herein can be used in any combination whether explicitly described or not, unless otherwise specified or clearly implied to the contrary by the context in which the referenced combination is made.

All combinations of method or process steps as used herein can be performed in any order, unless otherwise specified or clearly implied to the contrary by the context in which the referenced combination is made.

The compositions and methods of the present disclosure can comprise, consist of, or consist essentially of the essential elements and limitations of the composition and method described herein, as well as any additional or optional ingredients, components, or limitations described herein or otherwise useful in synthetic organic chemistry. The disclosure provided herein may be practiced in the absence of any element or step which is not specifically disclosed herein.

It is understood that the disclosure is not confined to the particular elements and method steps herein illustrated and described, but embraces such modified forms thereof as come within the scope of the claims.

### The CARM1 PROTACS

Disclosed herein are CARM1 PROTACs comprising a binding molecule configured to bind CARM1, a linker, and a ligand configured to bind an E3 ubiquitin ligase, wherein the binding molecule and the ligand are operationally linked by the linker. The E3 ligase ligand recruits the E3 ligase to CARM1. Upon ubiquitination, the cell removes CARM1 protein. The present disclosure shows that the CARM1 PROTACs developed herein can selectively degrade CARM1. The degradation of the protein leads to a reduction in mobility of cancer cells.

The CARM1 binding molecule, linker, and E3 ligase ligand are not limited to the exemplary embodiments described herein. Any binding molecules that bind CARM1 known in the art are contemplated to be useful herein. In certain versions, the binding molecule is a small molecule. An exemplary binding molecule provided herein is TP-064:

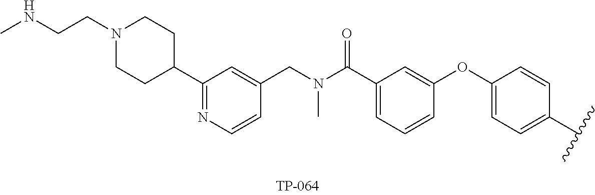 [SMILES: *c1ccc(Oc2cccc(C(=O)N(C)Cc3ccnc(C4CCN(CCNC)CC4)c3)c2)cc1]

The E3 ligase ligand may be a ligand of Von Hippel-Lindau (VHL) or cereblon (CRBN). Any VHL or CRBN ligands known in the art are contemplated to be useful herein. An exemplary E3 ligase ligand provide herein is a VHL ligand:

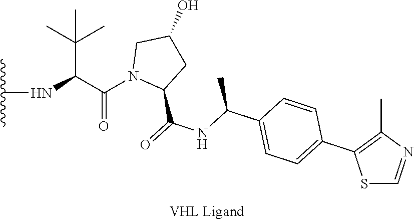 [SMILES: *N[C@H](C(=O)N1C[C@H](O)C[C@H]1C(=O)N[C@@H](C)c1ccc(-c2scnc2C)cc1)C(C)(C)C]

Thus, an exemplary CARM1 protein degrader provided herein comprises a CARM1 ligand TP-064, a linker, and a VHL E3 ligase ligand:

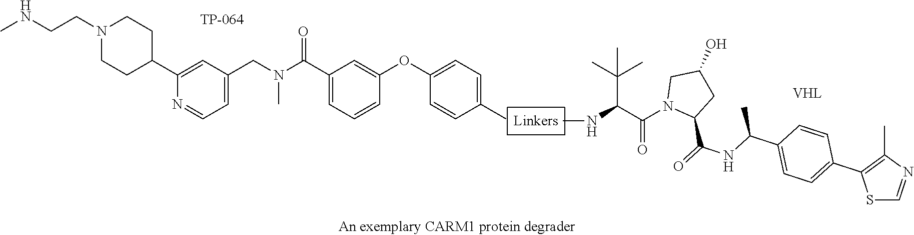

According to the disclosure, any desired linker can be used as long as the resulting compound is stable as part of a pharmaceutically acceptable dosage form, and itself is pharmaceutically acceptable.

In certain versions, the linker comprises stable and flexible alkyl linkages. An exemplary structure of the linker is as follows:

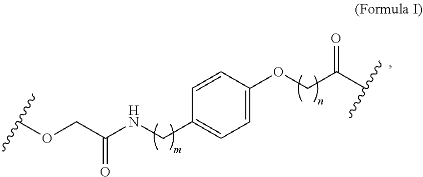

wherein m is 1 or 2; and n is an integer from 3 to 7.

In certain versions, the linker is a more rigid linker comprising heterocycles. An exemplary structure of the linker is as follows:

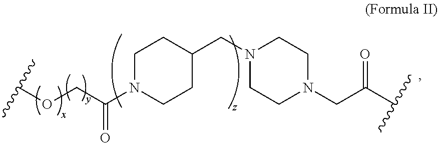

wherein x is 0 or 1; y is 0, 1, or 2; and z is 1 or 2.

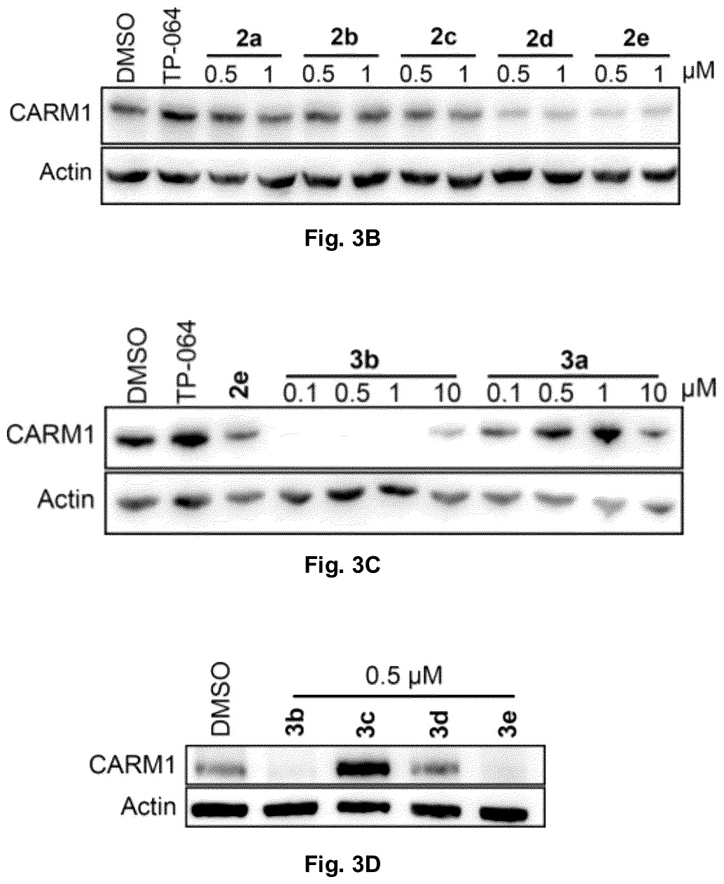

FIG. 3A shows non-limiting examples of alkyl linkers (2a-2e) and rigid linkers (3a-3e).

### Composition of Matter

The CARM1 PROTACs as disclosed herein can be administered as the neat chemical, but are more typically administered as a pharmaceutical composition, that includes an effective amount for a host, typically a human, in need of such treatment for any of the disorders described herein. Accordingly, the disclosure provides pharmaceutical compositions comprising an effective amount of the PROTAC or pharmaceutically acceptable salt together with at least one pharmaceutically acceptable carrier for any of the uses described herein. The pharmaceutical composition may contain a PROTAC or salt as the only active agent, or, in an alternative embodiment, the PROTAC and at least one additional active agent.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the PROTAC is sufficient to provide a practical quantity of material for administration per unit dose of the PROTAC.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the PROTAC of the present disclosure.

The pharmaceutical compositions according to the present disclosure suitable for administration to an individual (e.g., suitable for human administration) are generally sterile and may further be free of detectable pyrogens or other contaminants contraindicated for administration to an individual according to a selected route of administration.

For oral preparations, the PROTAC can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The PROTAC can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

### Method of Use

Disclosed herein are methods of using the PROTACs of the present disclosure.

Provided herein are methods of degrading CARM1 protein. Such methods include contacting the CARM1 protein with any of the PROTACs of the present disclosure, under conditions in which the PROTAC recruits an E3 ubiquitin ligase to CARM1. Such methods find use in a variety of applications. In certain aspects, the method is performed in vitro (e.g., in a tube, cell culture plate or well, or the like) and finds use, e.g., in testing and/or research applications. In other aspects, the method is performed in vivo (e.g., in an individual to whom the PROTAC is administered) and finds use, e.g., in clinical/therapeutic applications.

Also provided are methods that include administering to an individual in need thereof a therapeutically effective amount of any of the PROTACs or any of the pharmaceutical compositions of the present disclosure. A variety of individuals are treatable according to the subject methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

An effective amount of the PROTACs (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual (e.g., cancer) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the PROTACs or pharmaceutical composition.

The methods include administering to an individual having cancer a therapeutically effective amount of any of the PROTACs or any of the pharmaceutical compositions of the present disclosure. For example, the individual to be treated may have a cancer characterized by the presence of a solid tumor, a semi-solid tumor, a primary tumor, a metastatic tumor, or the like. In some embodiments, the individual has a cancer selected from breast cancer, melanoma, lung cancer, colorectal cancer, prostate cancer, glioma, bladder cancer, endometrial cancer, kidney cancer, leukemia (e.g., acute myeloid leukemia (AML)) liver cancer (e.g., hepatocellular carcinoma (HCC), such as primary or recurrent HCC), non-Hodgkin lymphoma, pancreatic cancer, thyroid cancer, any combinations thereof, and any sub-types thereof.

In any of the methods of using the PROTACs of the present disclosure, the PROTAC generally enhances degradation of the CARM1 protein relative to degradation of the protein in the presence of the protein binder alone. Similarly, in any of the methods of using the PROTACs of the present disclosure, according to some embodiments, the PROTACs enhances degradation of the CARM1 protein relative to degradation of the protein in the presence of the E3 ubiquitin ligase ligand alone.

By “treat”, “treating” or “treatment” is meant at least an amelioration of the symptoms associated with the medical condition (e.g., cell proliferative disorder, e.g., cancer) of the individual, where amelioration is used in a broad sense to refer to at least a reduction in the magnitude of a parameter, e.g., symptom, associated with the medical condition being treated. As such, treatment also includes situations where the medical condition, or at least symptoms associated therewith, are completely inhibited, e.g., prevented from happening, or stopped, e.g., terminated, such that the individual no longer suffers from the medical condition, or at least the symptoms that characterize the medical condition.

### Examples

### Summary

CARM1 is amplified or overexpressed in many cancer types and its overexpression correlates with poor prognosis. Potent small molecule inhibitors for CARM1 have been developed, but the cellular efficacy of the CARM1 inhibitors is limited. We herein report the development of the proteolysis targeting chimera (PROTAC) for CARM1 which contains a CARM1 ligand TP-064, a linker, and a VHL E3 ligase ligand. Compound 3b elicited potent cellular degradation activity (DC₅₀=8 nM and Dₘₐₓ>95%) in a few hours. Compound 3b degraded CARM1 in VHL- and proteasome-dependent manner and was highly selective for CARM1 over other protein arginine methyltransferases. CARM1 degradation by 3b resulted in potent downregulation of CARM1 substrate methylation and inhibition of cancer cell migration in cell-based assays. Thus, CARM1 PROTACs can be used to interrogate CARM1's cellular functions and potentially be developed as therapeutic agents for targeting CARM1-drived cancers.

### Results and Discussion

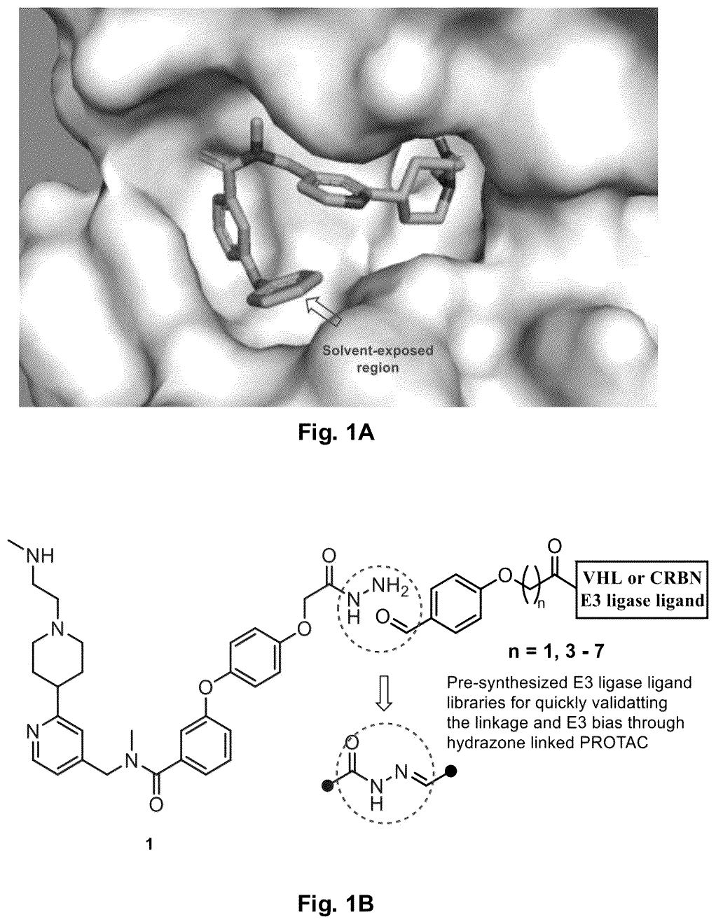

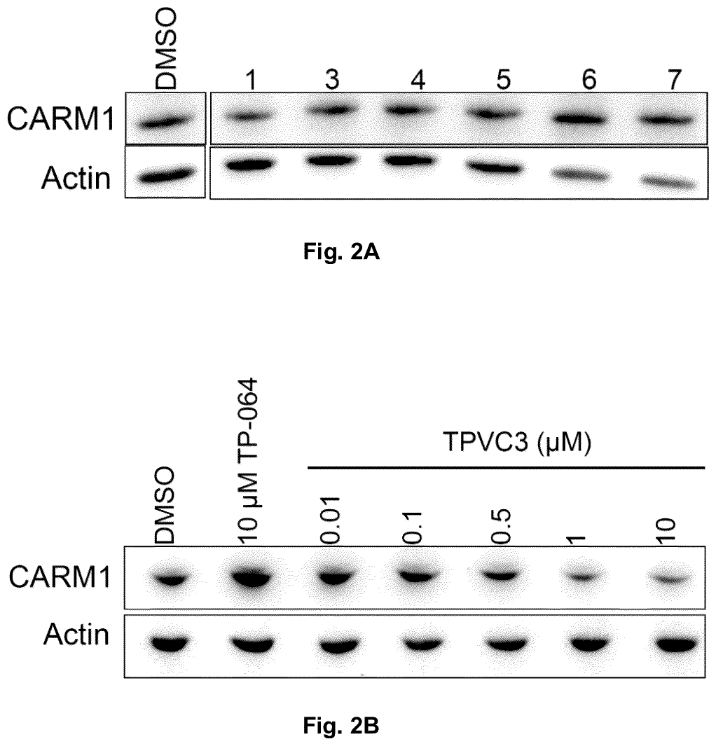

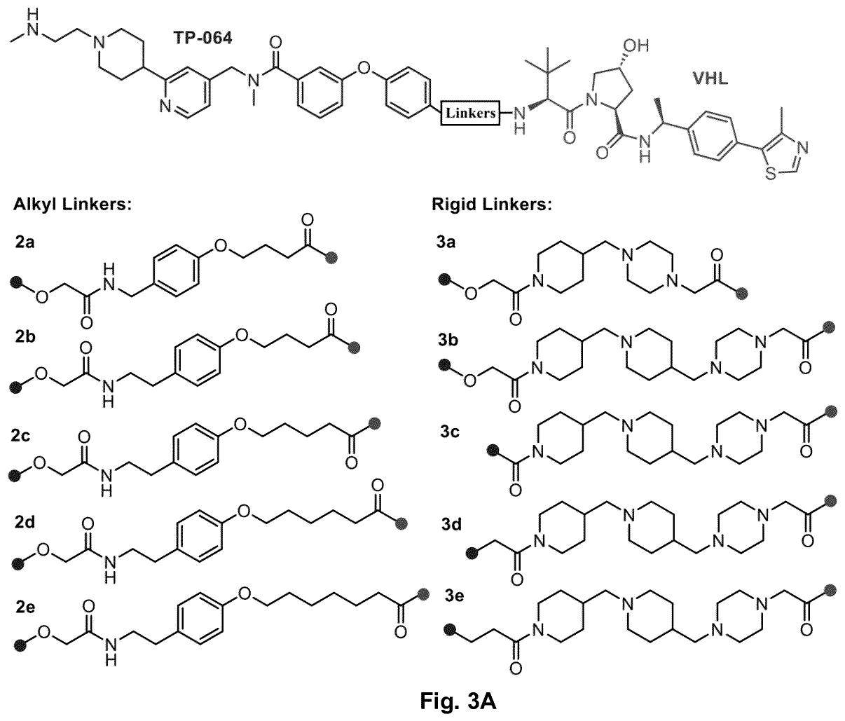

We designed the CARM1 PROTAC based on TP-064, a CARM1 small molecule inhibitor because TP-064 has a strong binding affinity and high selectivity for CARM1 (Nakayama et al. “TP-064, a Potent and Selective Small Molecule Inhibitor of PRMT4 for Multiple Myeloma” Oncotarget 2018, 9 (26), 18480-18493). To quickly identify the appropriate E3 ubiquitin ligase and the approximate linker length, we utilized a Rapid-TAC platform to generate the first set of PROTACs by coupling a hydrazide-containing CARM1 inhibitor with the aldehyde group in our pre-assembled PROTAC library. (See Roberts et al. “Two-Stage Strategy for Development of Proteolysis Targeting Chimeras and Its Application for Estrogen Receptor Degraders” ACS Chem. Biol. 2020, 15 (6), 1487-1496; Guo et al. “Platform for the Rapid Synthesis of Proteolysis Targeting Chimeras (Rapid-TAC) under Miniaturized Conditions” Eur. J. Med. Chem. 2022, 236, 114317; Guo et al. “Development of Selective FGFR1 Degraders Using a Rapid Synthesis of Proteolysis Targeting Chimera (Rapid-TAC) Platform” Bioorg. Med. Chem. Lett. 2022, 75, 128982.) After examining the crystal structure of CARM1 in complex with TP-064, the hydrazide moiety was placed on the para position of the terminal benzene, which is one of the solvent-exposed regions (FIGS. 1A and 1B). Compound 1, TP-064 hydrazide derivative, was then prepared. Six VHL ligand-based and six CRBN ligand-based PROTACs were quickly prepared in DMSO under miniaturized conditions. These PROTACs were tested in MCF7 cells at single concentrations (10 μM) for their ability to degrade CARM1. Western blot analysis revealed that VHL ligand based PROTACs induced obvious degradation of CARM1 in MCF7 cells (FIG. 2A). A dose-response curve was generated for TPVC3 (VHL ligand and n=3), the most potent degrader. Significant CARM1 degradation was observed at 1.0 and 10 μM (FIG. 2B).

Based on the structure of TPVC3, CARM1 degraders with stable and flexible alkyl linkages 2a-e were designed and synthesized. Degrader 2b bears the same linker length as TPVC3, while 2a has one less carbon. However, we also changed the hydrolytic labile C═N double bond, which is part of the acylhydrazone motif, in TPVC3 to a C—C bond in compound 2b. This change may influence the required optimal linker length. To cover a wider range of linkers, we next prepared degraders 2c, 2d, and 2e, which possess one, two, and three more carbon atoms, respectively, than compound 2b (FIG. 3A). Western blot assays were performed to evaluate the CARM1 degradation activity of the compounds after treating MCF7 cells with these degraders at two concentrations (0.5 and 1.0 μM) for 24 h. Compounds 2d and 2e showed stronger CARM1 degradation activities than 2a, 2b, and 2c (FIG. 3B). Based on the structure-activity relationship (SAR) of these CARM1 degraders with alkyl linkages, we next designed and prepared a series of degraders with more rigid linkers to further improve the potency and potentially other pharmacological properties. Compound 3a has two heterocycles including a piperazine ring and a piperidine ring. It has one less carbon atom than 2a in terms of length. Compound 3b has one piperazine ring and two piperidine rings. It has the same linker length as 2d. MCF7 cells were then treated with 3a and 3b at four concentrations (0.1, 0.5, 1.0, and 10.0 μM) for 24 hours and compared with 2e, the best compound with a flexible linker, at 0.5 μM. Western blot results showed that 3b possessed significantly improved CARM1 degradation activity (FIG. 3C). To fine-tune the linker length and to avoid the potential metabolic liability of having two para-substituted oxygen atoms on one benzene ring, compounds 3c, 3d, and 3e were designed and synthesized. The terminal phenyl group in TP-064 was conjugated with piperidine by benzamide, benzeneacetamide, and 3-phenylpropanamide groups, respectively (FIG. 3A). After treating MCF7 cells with 0.5 μM of degraders 3b, 3c, 3d, or 3e, for 24 h, compounds 3b and 3e showed similar CARM1 degradation activity as they have the same linker length. Interestingly, compounds 3d and 3c, which bear one and two less carbon atoms, respectively, than 3e, displayed significantly lower CARM1 degradation activities. The sharp change of CARM1 degradation activity with just two atom difference between compounds 3c and 3b or compounds 3c and 3e (FIG. 3D) is striking, indicating that the linker is critical for positioning CARM1 to the proteasome for degradation.

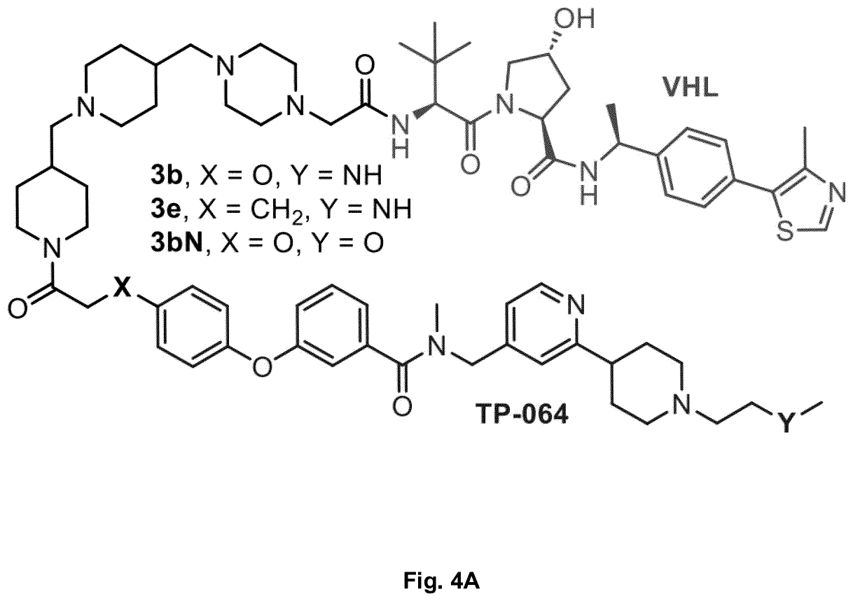

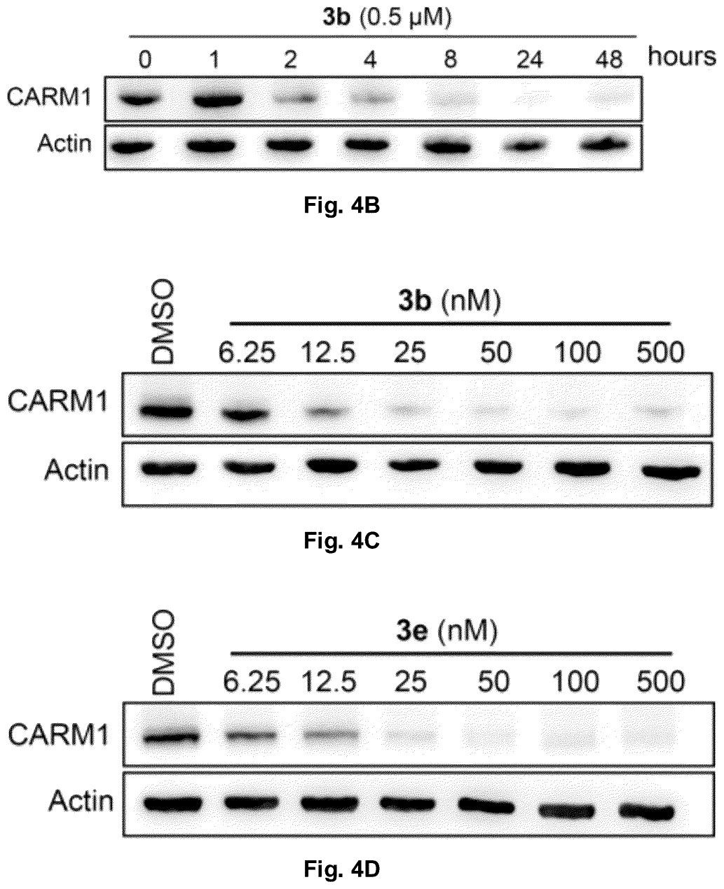

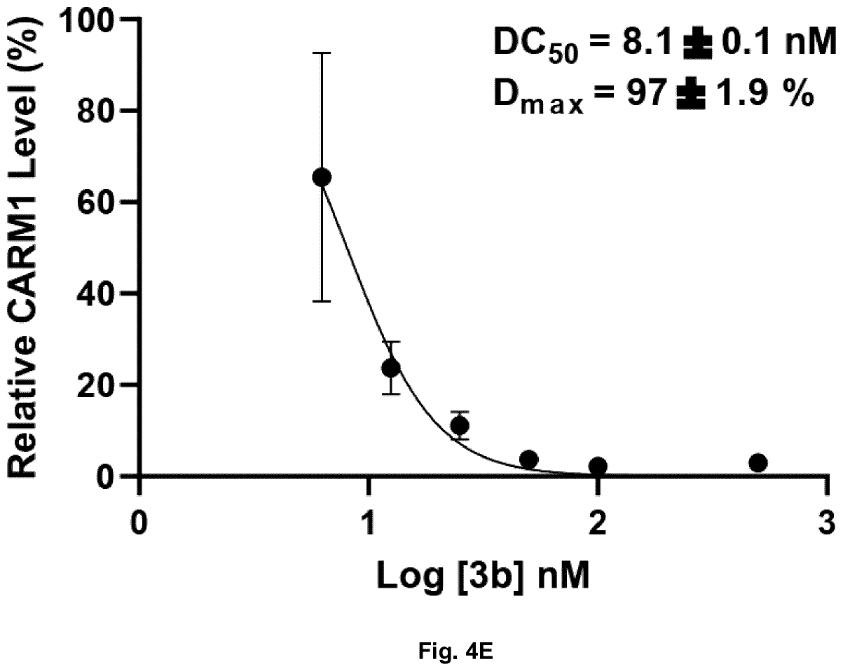

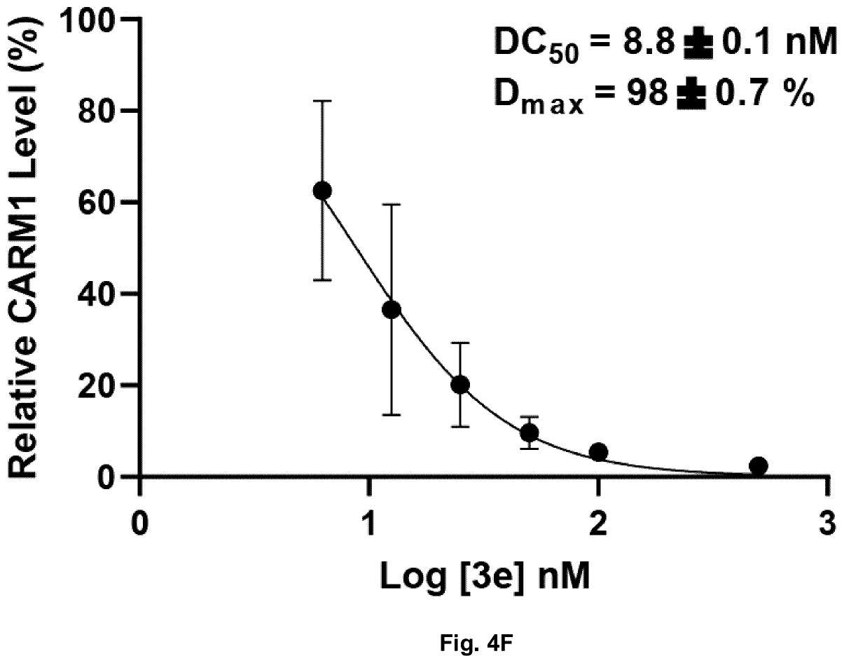

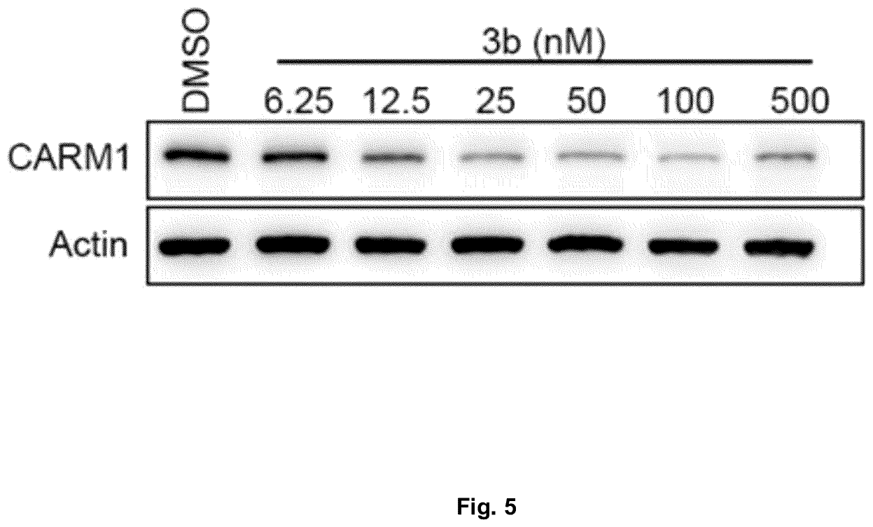

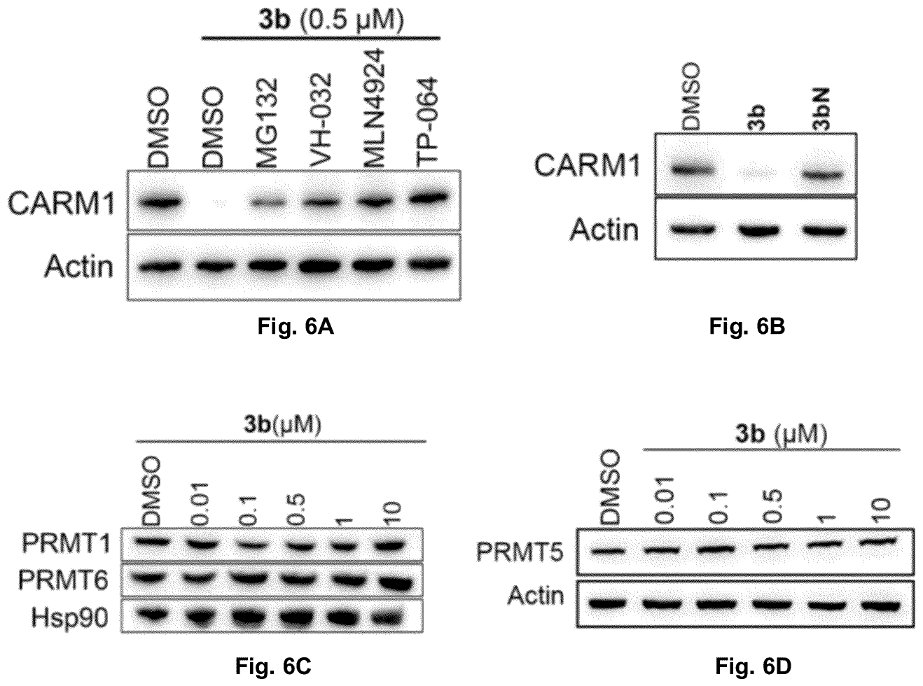

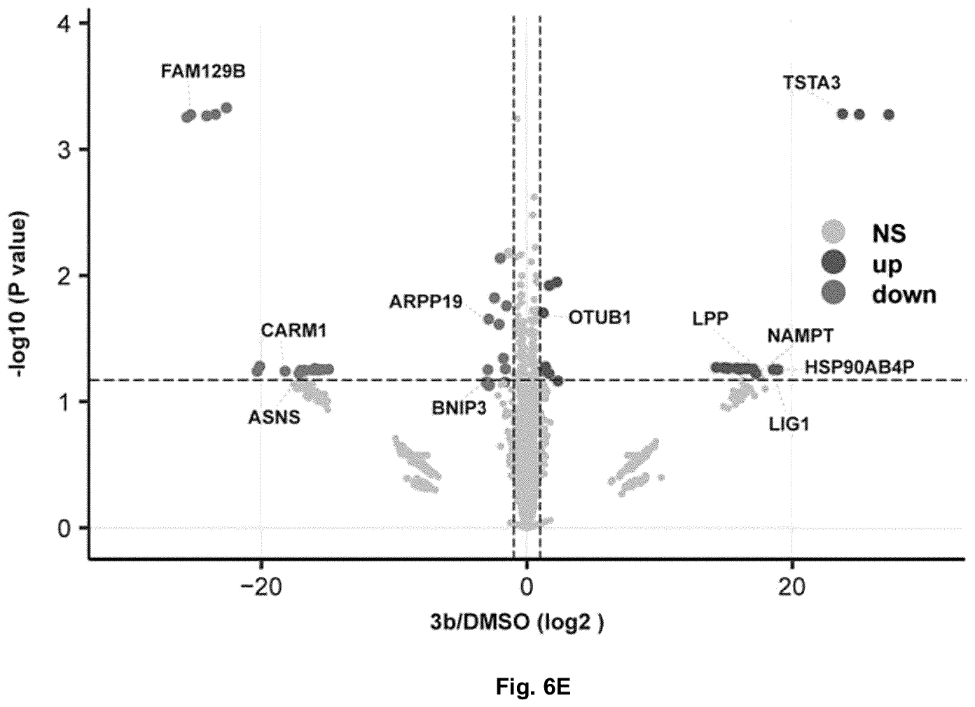

The most potent degraders 3b and 3e were then selected for further evaluation (FIG. 4A). We performed a time course study for CARM1 degrader 3b. CARM1 degradation was observed as soon as 2 h after exposing to 3b, and degradation sustained and further maximized over the course of 48 h (FIG. 4B). Next, dose-response curves were measured for degraders 3b and 3e. In MCF7 cells, 3b and 3e showed high potency for CARM1 degradation, with DC₅₀ and Dₘₐₓ of 8.1±0.1 nM, 97±1.9% and 8.8±0.1 nM, 98±0.7%, respectively (FIGS. 4C-4F). Compound 3b also showed high potency for CARM1 degradation in MCF10A cells, a normal human mammary epithelial cell model (FIG. 5). We then validated the mechanisms of action of these degraders using competitive inhibitors of CRL2^{VHL} ligase complex and the 26S proteasome (FIG. 6A). Pretreatment of the MCF7 cells with CARM1 inhibitor TP-064, VHL ligand VH-032, the proteasome inhibitor MG132, or neddylation inhibitor MLN492437 all abrogated 3b-induced CARM1 degradation, confirming that CARM1 degradation by 3b engages CARM1, VHL, the proteasome, and the Cullin-RING E3 ligase complex. We also prepared 3bN, a negative control compound, which cannot bind to CARM1 due to the lack of secondary amine (Y═O instead of NH) as reported in the literature (Nakayama et al. “TP-064, a Potent and Selective Small Molecule Inhibitor of PRMT4 for Multiple Myeloma” Oncotarget 2018, 9 (26), 18480-18493). No CARM1 degradation was observed for 3bN in MCF7 cells (FIG. 6B). To evaluate the selectivity of degrader 3b, PRMT1, PRMT6, and PRMT5 levels were measured by Western blot. In contrast to 3b induced CARM1 degradation with an 8.1 nM of DC₅₀ and 97% of Dₘₐₓ (FIG. 4C), PRMT1, PRMT6, and PRMT5 degradation were not detected in MCF7 cells under the same conditions (FIGS. 6C-6D). In order to systematically assess the alternations in the proteome caused by 3b, we performed a global quantitative proteomic analysis of MCF7 cells using mass spectrometry. The cells were treated with DMSO, 25 nM 3b or 3bN for 4 h. The treatment dose and time were determined based on time-course and dose titration experiments (FIG. 4B-4C). In total, 3491 proteins were quantified among three cohorts. Only 45 and 25 significantly changed proteins were identified in the 3bN and 3b groups as compared to the DMSO group with two-fold cutoff, respectively (p-value ≤0.05). The protein CARM1 demonstrated significant downregulation specifically in the 3b group and not in the 3bN group (FIG. 6D). Two other PRMTs, PRMT1, and PRMT5, were detected in proteomics analyses but their levels were not significantly changed by 3b, reinforcing the selectivity of 3b to CARM1. Notably, several proteins (e.g., FAM129B) were also found to be significantly downregulated, which aligns with previous findings in CARM1 KO cells (Shishkova et al. “Global Mapping of CARM1 Substrates Defines Enzyme Specificity and Substrate Recognition” Nat. Commun. 2017, 8 (1), 15571). This suggests that these proteins are likely associated with the functions regulated by CARM1.

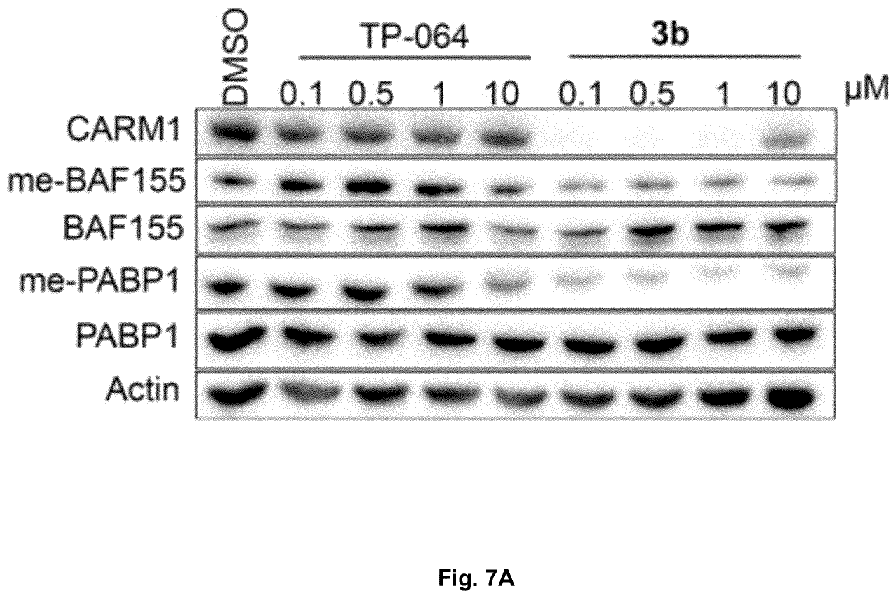

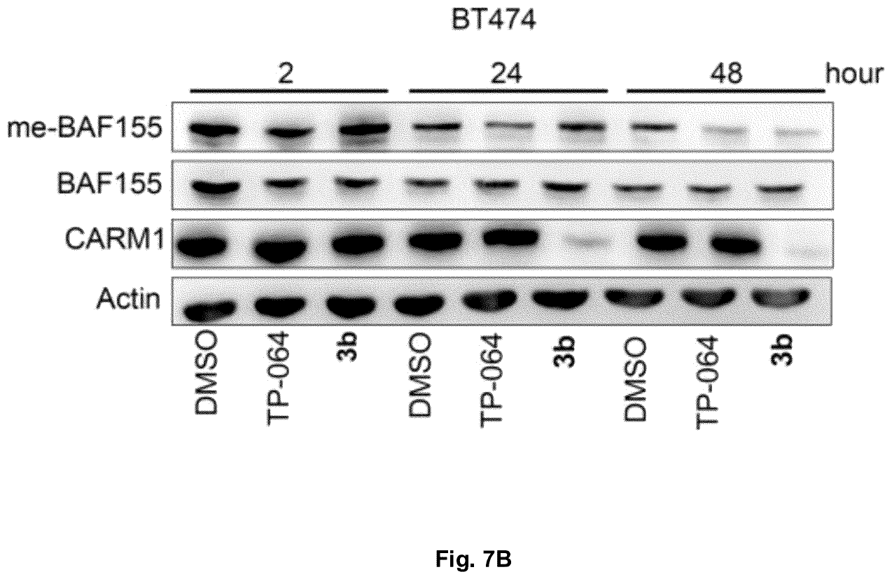

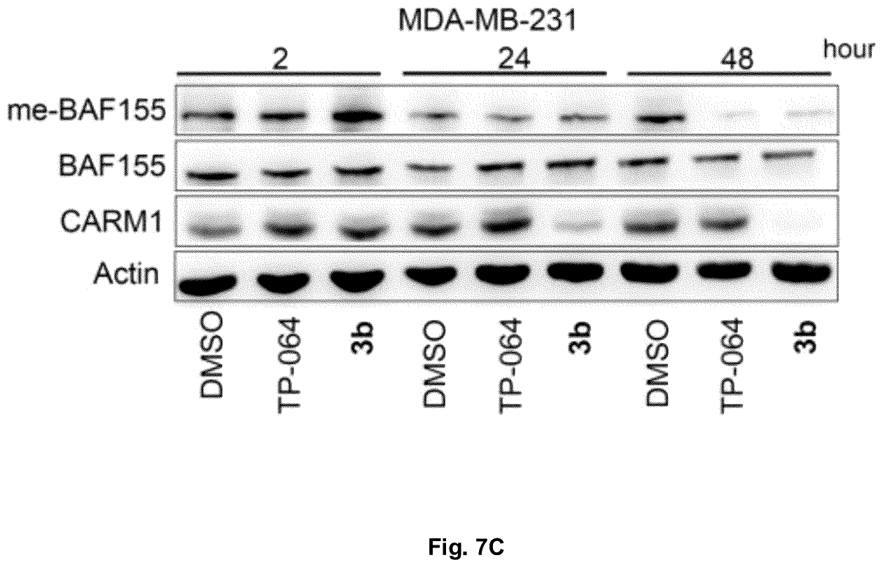

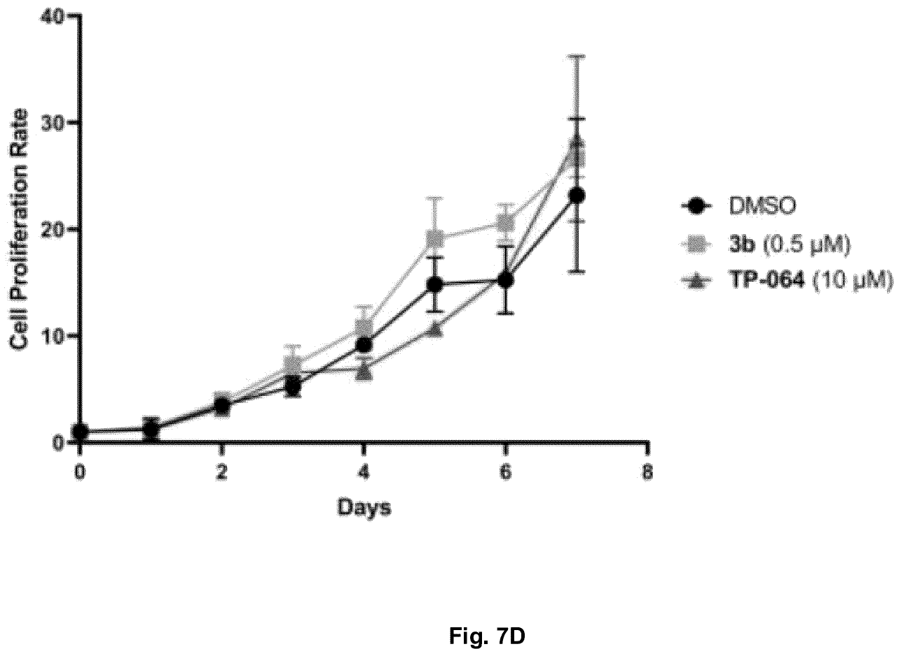

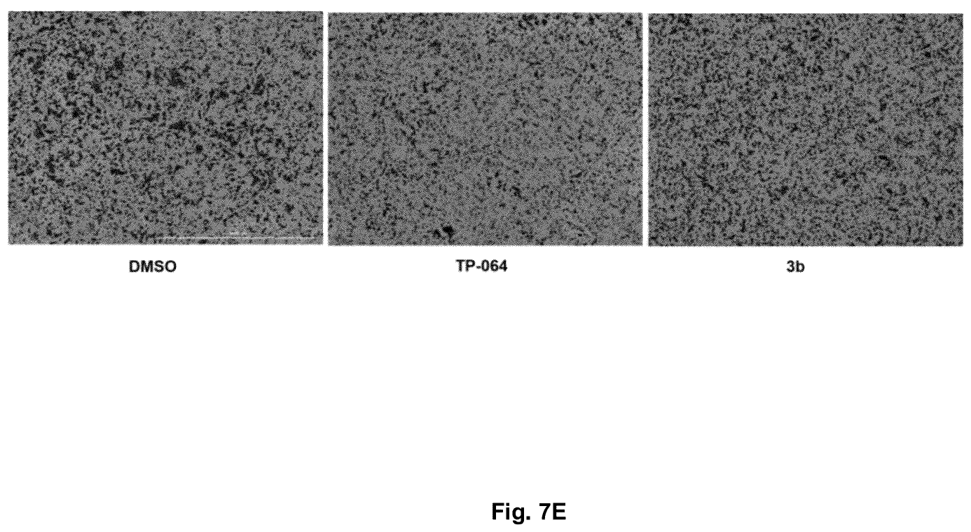

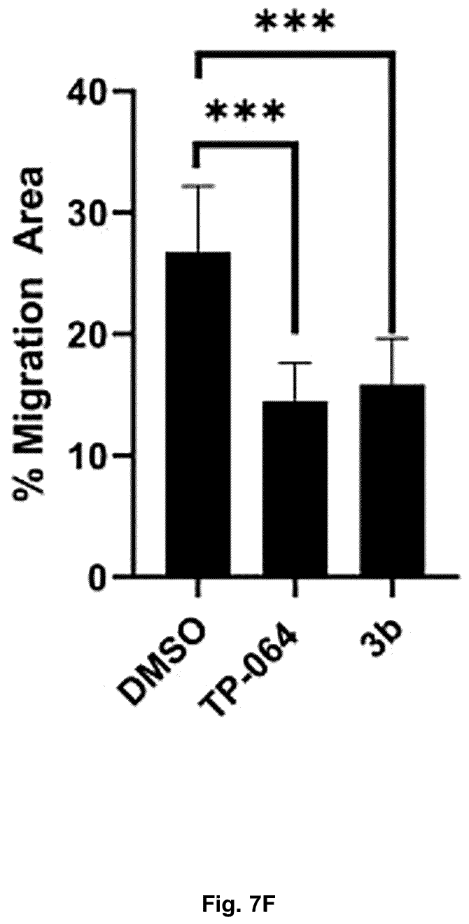

To demonstrate the effectiveness of 3b to inhibit cellular protein methylation by CARM1, we selected CARM1-specific substrates Polyadenylate-binding protein 1 (PABP1) and BRG1-associated factor 155 (BAF155) for analyses because their methyl-specific antibodies are available. (See Wang et al. “CARM1 Methylates Chromatin Remodeling Factor BAF155 to Enhance Tumor Progression and Metastasis” Cancer Cell 2014, 25 (1), 21-36; Lee and Bedford. “PABP1 Identified as an Arginine Methyltransferase Substrate Using High-Density Protein Arrays” EMBO Rep. 2002, 3 (3), 268-273.) In fact, asymmetric dimethylation (me2a) of these proteins are routinely investigated for CARM1 inhibitors. Herein, the PABP1 and BAF155 di-methylation inhibitory effects in MCF7 cells by CARM1 inhibitor TP-064 and degrader 3b were compared side-by-side. As shown in FIG. 7A, 3b is at least 100-fold more potent than TP-064 in terms of functional outcome. The levels of PABP1 and BAF155 me2a inhibition with 0.1 μM 3b are at least as equivalent to that of 10 μM of TP-064 (Kim et al. “BAF155 Methylation Drives Metastasis by Hijacking Super-Enhancers and Subverting Anti-Tumor Immunity” Nucleic Acids Res. 2021, 49 (21), 12211-12233). The CARM1 degradation activity of 3b is not restricted to MCF7 cells, as CARM1 was also effectively degraded by 3b after 24 or 48 h in ER+/HER2+ BT474 and triple-negative MDA-MB-231 breast cancer cell lines. The decrease of BAF155 me2a level was observed after 48 h in both cell lines in TP-064 or 3b treated groups (FIGS. 7B and 7C).

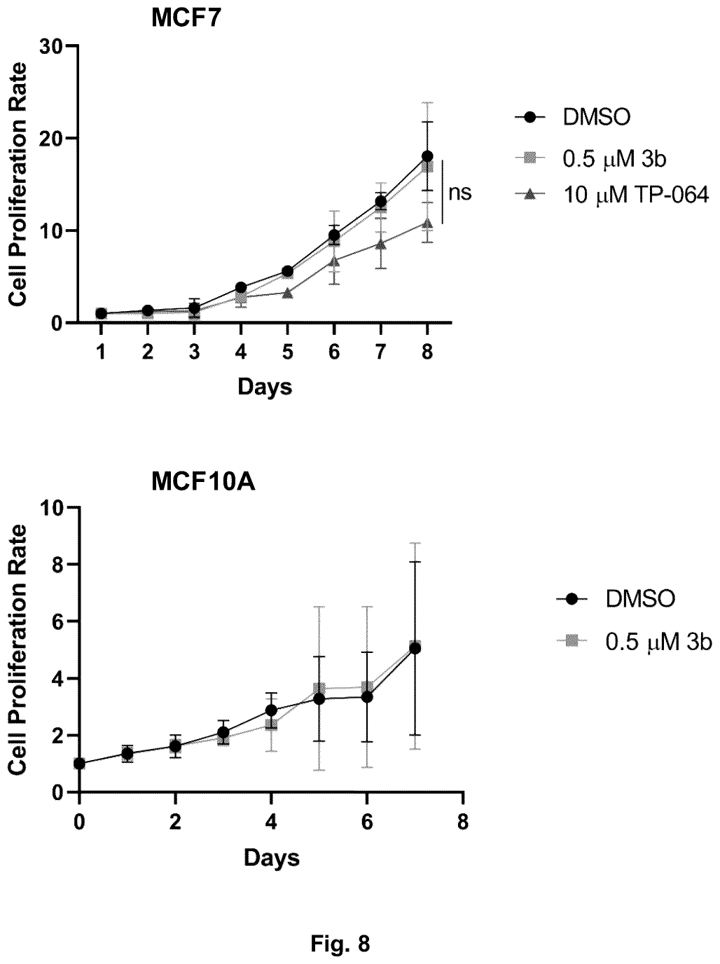

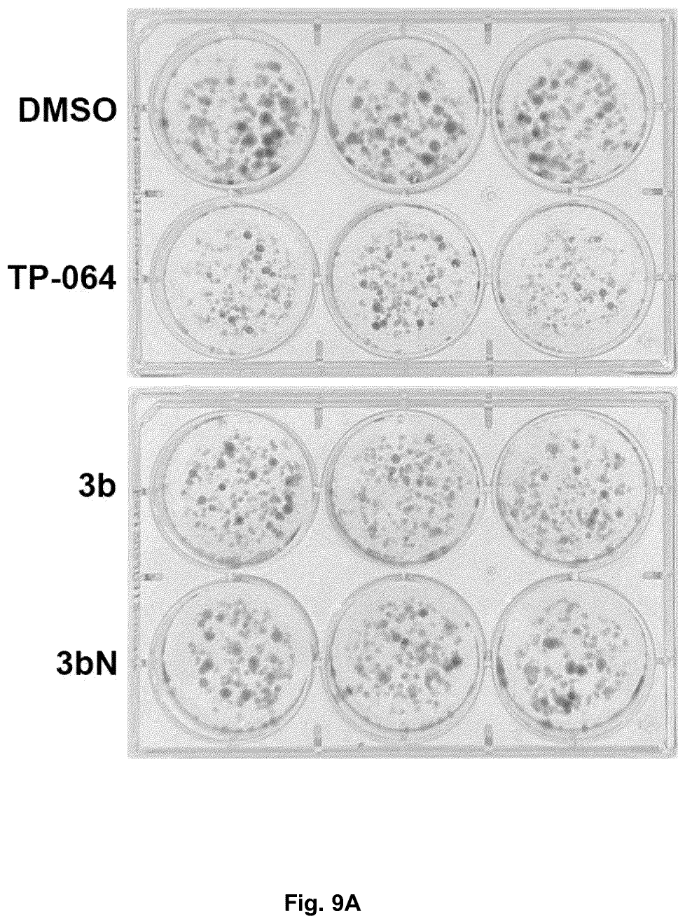

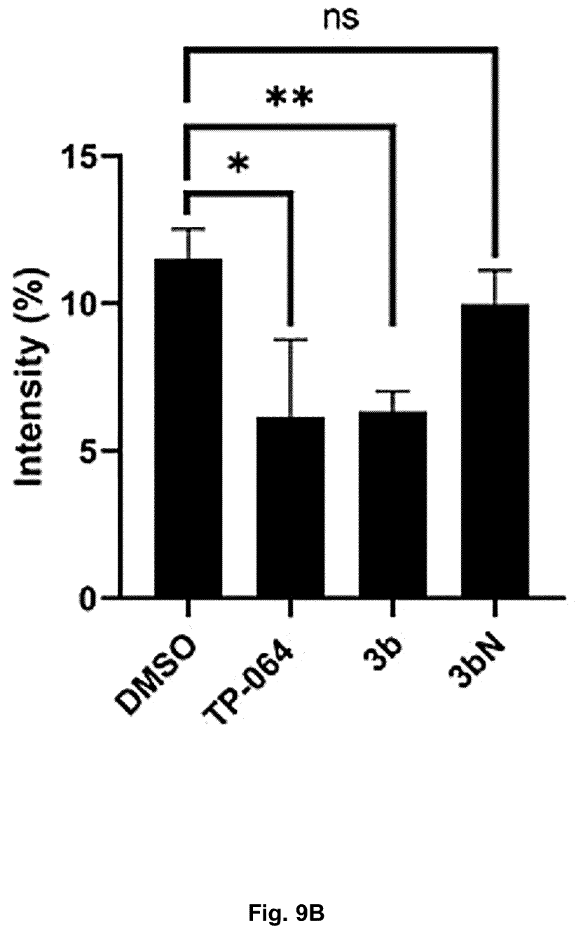

After confirming that 3b induces CARM1 degradation and inhibits CARM1 downstream enzymatic effect in different breast cancer cell lines representing three breast cancer subtypes, the effects of 3b on cell proliferation and migration were determined. CARM1 inhibitors were reported to have low cytotoxic effects in breast cancer cells, yet they significantly inhibited cell migration, an indicator of metastasis (Kim et al. “BAF155 Methylation Drives Metastasis by Hijacking Super-Enhancers and Subverting Anti-Tumor Immunity” Nucleic Acids Res. 2021, 49 (21), 12211-12233). Similarly, 3b was found not to inhibit cell proliferation in normal mammary epithelial cell line MCF10A and breast cancer cell lines MCF7 and MDA-MB-231 cells (FIGS. 7D and 8). Because ER-positive breast cancer cells have low migratory effects and CARM1 is highly expressed in triple-negative breast cancer cells, we measured the effects of 3b on cell migration using a transwell migration assay for MDA-MB-231 cells. We found that 10 μM of TP-064 and 0.5 μM of 3b treatment inhibited cell migration at a similar level (FIGS. 7E and 7F). Compound 3b can induce the same anti-migratory effects with a 20-fold lower dose compared to the corresponding inhibitor, indicating that CARM1 degrader can be much more potent than the corresponding inhibitor in cell-based assays for eliciting the same biological effects. The results were confirmed by a colony formation assay, wherein MDA-MB-231 were seeded 200 cells per well and treated with DMSO, 10 μM TP-064, 0.5 μM 3b and 3bN for two weeks. After the treatment period, cells were fixed and stained with crystal violet. The quantification of the intensity of the staining is graphed in FIGS. 9A and 9B.

The synthesis of CARM1 binders S2-S5 and a negative control S6 is summarized in Scheme 1. Secondary amine S1b is a common building block for the preparation of the right half of compounds S2-S6. The left half of compounds S2-S6 are derived from carboxylic acid derivatives S2c, S3a, S4c, and S5c. First, cross-coupling of boronic ester with a bromo pyridine derivative afforded intermediate S1a, which can then undergo reductive amination to yield amine S1b. Carboxylic acid S2c was prepared from p-iodophenol in four steps through standard alkylation, cross-coupling, saponification followed by acidification. Acid S3a was prepared by nucleophilic aromatic substitution (S_{N}Ar) reaction. Acid S4c was prepared in four steps through the sequence of S_{N}Ar, reduction, saponification followed by esterification. Finally, acid S5c was prepared by S_{N}Ar, olefination, saponification followed by esterification. The coupling of carboxylic acids S2c, S3a, S4c, and S5c with amine S1b afforded the corresponding amides, which can then undergo de-protection followed by reductive amination to yield key building blocks S2-S6.

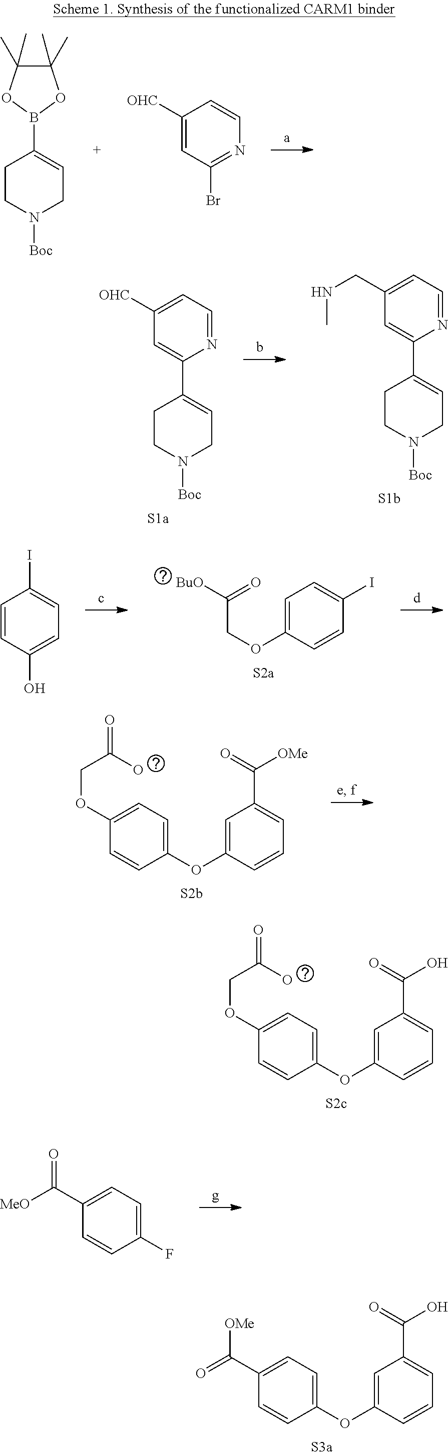

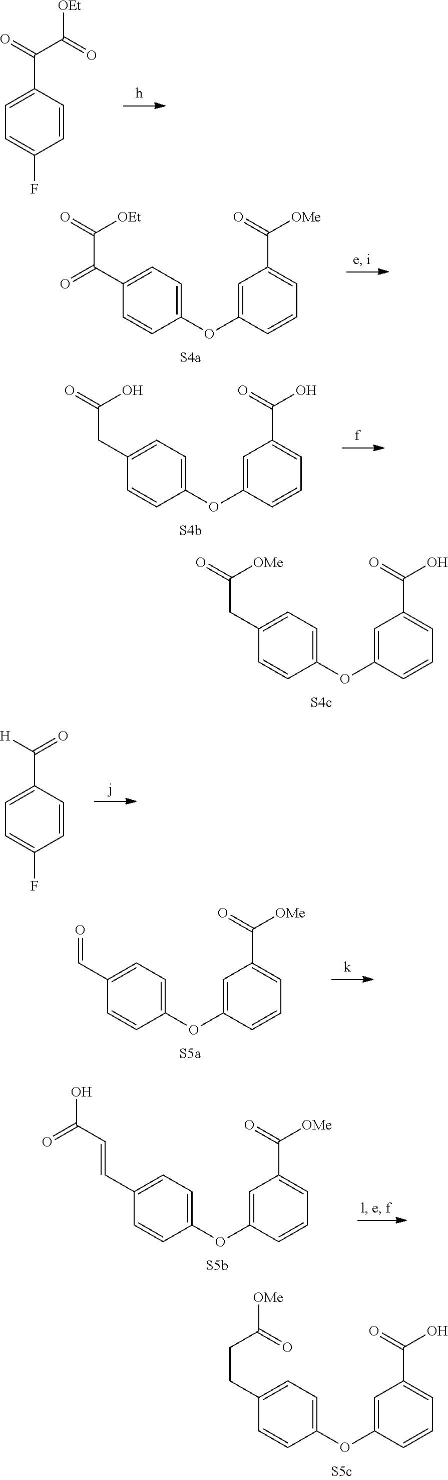

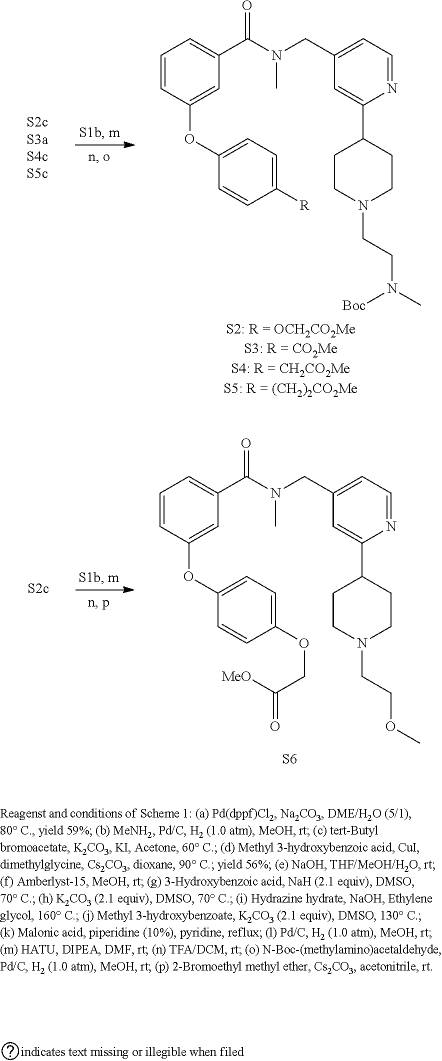

The synthesis of CARM1 PROTACs is summarized in Scheme 2. First, linkers AL1+3 to AL2+6 were prepared by Boc-protection and alkylation from phenols with a remote amino group linked by different number of methylenes. Next, piperidine- and piperazine-containing rigid linkers RL1 and RL2 were prepared by alkylation followed by deprotection and reductive elimination. Commercially available ligand Boc-VHL, linker AL1+3, and key intermediate S2 were linked together to form CARM1 PROTAC 2a via amide formation reactions followed by de-protection of the Boc-group. All other CARM1 PROTACs were prepared similarly by simply coupling the three pieces through amide bond formation.

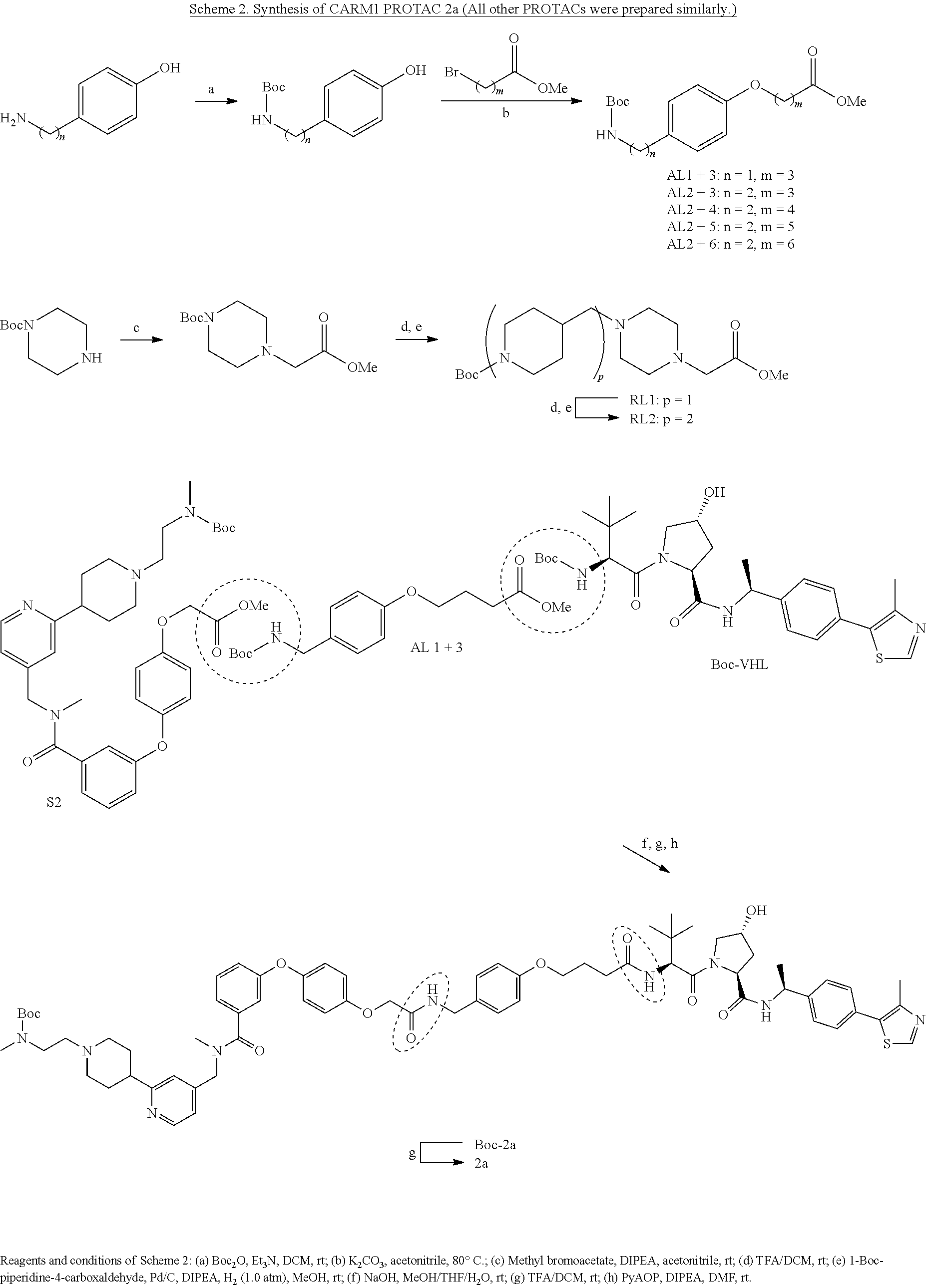

In summary, potent and selective CARM1 PROTACs were developed in this Example. Appropriate E3 ubiquitin ligase and linker length were quickly identified using the Rapid-TAC platform. Further optimization of the linker by replacing the flexible alkyl group with rigid piperidine and piperazine rings yielded potent PROTACs 3b and 3e with DC₅₀ around 8 nM and Dₘₐₓ over 95%. We are able to achieve more significant downstream effects (i.e., methylation of the substrates) using 100 times less concentration of the degrader than the inhibitor. Functional studies indicated that CARM1 PROTACs could potently inhibit the migration of breast cancer cells. Further study will be conducted to investigate the non-enzymatic effect of CARM1 by comparing the CARM1 degrader and inhibitor.

### Experimental Sections

Western Blot Assay. Cells were harvested and washed with Dulbecco's phosphate-buffered saline (PBS), then lysed with 1×RIPA lysis buffer (50 mM Tris pH 7.5, 150 mM NaCl, 1% NP-40, 0.5% sodium deoxycholate, and 0.1% sodium dodecyl sulfate) supplemented with 1 mM phenylmethylsulfonyl fluoride, 10 μg/mL aprotinin, 1 μM leupeptin, and 10 μg/mL pepstatin on ice for 20 min. The supernatant was collected after spinning down at 15000 RPM at 4 degrees Celsius for 10 min. The concentration of whole lysates was quantified using a Bradford assay. 50-75 μg of cell lysates was boiled in 4× Laemmli Loading Dye and boiled at 100 degrees Celsius for 10 min. 25 μg were run in 8% SDS-PAGE gels and transferred to nitrocellulose membranes. Blots were blocked in 5% skim milk in PBST [PBS and 0.1% Tween 20] for 45 min, then incubated with primary antibodies at 4 degrees Celsius overnight. Blots were washed in PBST for 30 min, then incubated with horseradish peroxidase-linked secondary antibodies (Jackson ImmunoResearch) for 1 hr. Afterwards, blots were washed in PBST for 15-30 min, then treated with chemiluminescent ECL reagent and imaged with the ChemiDoc Imaging System (Bio-Rad) or Azure 600 (Azure Biosystems). Expression levels of the indicated proteins were probed by the following antibodies: anti-CARM1, anti-me-BAF155, anti-me-PABP1, anti-PABP1,⁷ anti-BAF155 (D7F8S, Cell Signaling Technology), anti-PRMT1 (Bethyl), anti-PRMT6 (D-5, Santa Cruz Biotechnology), anti-β-Actin (ABclonal Technology, Sigma-Aldrich), anti-Hsp90 (H-114, Santa Cruz Biotechnology).

Sample preparation for global proteomics analysis. Sample preparation is similar to the previous report (Ma et al. “Strategy Based on Deglycosylation, Multiprotease, and Hydrophilic Interaction Chromatography for Large-Scale Profiling of Protein Methylation” Anal. Chem. 2017, 89 (23), 12909-12917). Briefly, treated MCF7 cells were harvested by washing with PBS buffer (Gibco, pH 7.4) and lysed in RIPA buffer supplemented with protease inhibitors for 20 min, then protein concentration was determined using a BCA Protein Assay Kit (Thermo Pierce, Rockford, IL) per manufacturer instructions. Samples were reduced with 5 mM DTT for 1 hr and alkylated with 10 mM IAA for 30 min in the dark before quenching with 5 mM DTT. Proteins were digested by trypsin at 37° C. for 16 hours in a 50:1 (protein:enzyme) ratio. Digests were quenched by lowering the pH to <3 with 10% TFA. Peptides were desalted with SepPak C18 solid-phase extraction (SPE) cartridges (Waters, Milford, MA). The concentrations of the peptide mixture were measured by peptide assay (Thermo Fisher Scientific). All samples were dried in vacuo and stored at −80° C. until LC-ESI-MS/MS analysis.

LC-MS/MS data acquisition. Samples were analyzed on an Orbitrap Fusion Lumos Tribrid mass spectrometer (Thermo Fisher Scientific) coupled to a Dionex UltiMate 3000 UPLC system. Each sample was dissolved in 0.1% formic acid in water before being loaded onto a 75 μm inner diameter homemade microcapillary column that is packed with 15 cm of Bridged Ethylene Hybrid C18 particles (1.7 μm, 130 Å, Waters) and fabricated with an integrated emitter tip. Mobile phase A was composed of water and 0.1% formic acid, while mobile phase B was composed of ACN and 0.1% formic acid. LC separation was achieved across a 137-min gradient elution of 3-30% mobile phase B at a flow rate of 300 nL/min. Survey scans of peptide precursors from m/z 300 to 1500 were performed at a resolving power of 60 k (at m/z 200) with an AGC target of 2×10⁵ and maximum injection time of 100 ms. The top 20 precursors were then selected for higher energy collisional dissociation fragmentation with a normalized collision energy of 30, an isolation width of 1.0 Da, a resolving power of 15 k, and an AGC target of 1×10⁴. Precursors were subject to dynamic exclusion for 45 s with a 10 ppm tolerance. Each sample was acquired in technical triplicates.

Data analysis. Protein identification and quantification by MaxQuant (version 1.5.3.8) based database searching, using the integrated Andromeda search engine with FDR <1% at peptide and protein levels. The tandem mass spectra were searched against the Homo sapiens reviewed database (version updated December 2023). A reverse database for the decoy search was generated automatically in MaxQuant. Enzyme specificity was set to ‘Trypsin/p’, and a minimum number of seven amino acids were required for peptide identification. For label-free protein quantification (LFQ), the MaxQuant LFQ algorithm was used to quantitate the MS signals, and the proteins' intensities were represented in LFQintesnit (Schaab et al. “Analysis of High Accuracy, Quantitative Proteomics Data in the MaxQB Database” Mol. Cell. Proteomics 2012, 11 (3), M111.014068). Cysteine carbamidomethylation was set as the fixed modification. The oxidation of M and acetylation of the protein N-terminal were set as variable modifications. The first search mass tolerance was 20 ppm, and the main search peptide tolerance was 6 ppm. The false discovery rates of the peptide-spectrum match and proteins were set to less than 1%. For peptide quantification, the intensities of all samples were extracted from the MaxQuant result peptide files. Then, the expression matrix was subjected to normalization followed by log 2-transformed by Perseus (Tyanova et al. “The Perseus Computational Platform for Comprehensive Analysis of (Prote)Omics Data” Nat. Methods 2016, 13 (9), 731-740). From three technical replicates, the mean protein intensities were calculated for each biological replicate and subjected to statistical analysis. Bioinformatic analyses were performed with R software environment.

Cell Proliferation Assay. Cell proliferation was measured by direct cell count via high contrast brightfield microscopy. MDA-MB-231 cells (3×10³/well) were seeded in 100 μL of DMEM into the wells of a 96-well plate. Cells were treated with either DMSO, TP-064, or 3b and refreshed every two days. The plate was imaged daily for 7 days using the High-Contrast Brightfield Kit for Label-Free Cell Counting (BioTek) on the BioTek Imager.

Transwell Migration Assay. Cells were harvested and washed in serum-free DMEM. The wells of a 24-well plate were filled with 800 μL of DMEM with 10% FBS. Transwell inserts with 8.0 μM pore size were then added to the wells with DMEM. Cells were resuspended in serum-free DMEM, then 1×10⁵ cells in 200 μL were added to the upper chambers. Drug treatments were added to the lower chambers, then incubated for 20 h at 37° C. The cells on the upper surface were removed with cotton swabs. Migratory cells were fixed in 3.7% formaldehyde at room temperature, followed by 100% methanol at −20° C. Fixed cells were stained with 1% crystal violet in 20% methanol for 30 min. The migration area was quantified from five independent fields of view of the lower surface of the membrane under a microscope.

### General Information in Synthetic Chemistry

All reactions were conducted under a positive pressure of dry argon in glassware that had been oven-dried prior to use. Anhydrous solutions of reaction mixtures were transferred via an oven-dried syringe or cannula. All solvents were dried prior to use unless noted. Thin-layer chromatography (TLC) was performed using precoated silica gel plates. Flash column chromatography was performed with silica gel. ¹H and ¹³C nuclear magnetic resonance (NMR) spectra were recorded on Bruker 400, 500, 600 MHz and Varian 500 MHz spectrometers. ¹H NMR spectra were reported in parts per million (ppm) referenced to 7.26 ppm of CDCl₃ or referenced to the center line of a septet at 2.50 ppm of DMSO-d₆. Signal splitting patterns were described as singlet(s), doublet (d), triplet (t), quartet (q), quintet (quint), or multiplet (m), with coupling constants (J) in hertz. High-resolution mass spectra (HRMS) were performed on an electron spray injection (ESI) TOF mass spectrometer.

The liquid chromatography-mass spectrometry (LC-MS) analysis of final products was processed on an Agilent 1290 Infinity II LC system using a Poroshell 120 EC-C18 column (5 cm×2.1 mm, 1.9 μm) for chromatographic separation. Agilent 6120 Quadrupole LC/MS with multimode electrospray ionization plus atmospheric pressure chemical ionization was used for detection. The mobile phases were 5.0% methanol and 0.1% formic acid in purified water (A) and 0.1% formic acid in methanol (B). The gradient was held at 5% (0-0.2 min), increased to 100% at 2.5 min, then held at isocratic 100% B for 0.4 min, and then immediately stepped back down to 5% for 0.1 min re-equilibration. The flow rate was set at 0.8 mL/min. The column temperature was set at 40° C. The purities of all of the final compounds were determined to be over 95% by LC-MS. See the Supporting Information for ¹H and ¹³C NMR spectra and LC-MS purity analysis of all compounds.

Procedure for the preparation of compound S1b. To a 250 mL flask with a magnetic stirring bar, commercially available N-Boc-1,2,3,6-tetrahydropyridine-4-boronic acid pinacol ester (6.0 g, 19.4 mmol), 2-bromopyridine-4-carboxaldehyde (2.4 g, 12.9 mmol), Pd (dppf) C12 (512 mg, 0.63 mmol), Na₂CO₃ (3.4 g, 31.5 mmol), were sequentially added, evacuated, and backfilled with argon. Dimethoxyethane (DME) (30 mL) and water (6 mL) were successively added using a syringe. The reaction mixture was heated to 80° C. until the starting material 2-bromopyridine-4-carboxaldehyde disappeared, as indicated by TLC (˜4 h). The mixture was partitioned between ethyl acetate and a saturated solution of sodium bicarbonate; the organic layer was washed with brine, dried over Na₂SO₄, and concentrated in vacuum. The residue was purified by column chromatography on silica to provide S1a as colorless oil. (2.2 g, yield of 59%).

A 250 mL flask was charged with a magnetic stirring bar and S1a (2.0 g, 6.94 mmol). Next, methanol (70 mL), palladium on carbon (10 wt. %) (0.4 g) and methylamine (40 wt. % solution in water) (1.4 mL) were added. A hydrogen balloon was connected with the reaction mixture through a needle. The reaction mixture was stirred at room temperature until the starting material S1a disappeared, as indicated by TLC. The mixture was filtered through Celite and washed with methanol (3×5 mL); the mixture was then concentrated in vacuum. The residue was purified by column chromatography on silica to provide S1b as colorless oil. (2.0 g, yield of 95%).

Procedure for the preparation of compound S2c. To a 100 mL flask with a magnetic stirring bar, 4-Iodophenol (4.4 g, 20.0 mmol), tert-butyl bromoacetate (3.25 mL, 22.0 mmol), K₂CO₃ (3.04 g, 22.0 mmol), acetone (20 mL) was sequentially added. The reaction mixture was heated to 60° C., until the starting material disappeared, as indicated by TLC. The mixture was partitioned between ethyl acetate and a saturated solution of sodium bicarbonate; the organic layer was washed with brine, dried over Na₂SO₄, and concentrated in vacuum. The residue was purified by column chromatography on silica to provide S2a as light-yellow solid. (6.4 g, yield of 95%).

In a 100 mL flask with a magnetic stirring bar, S2a (3.0 g, 9.0 mmol), methyl 3-hydroxybenzoic acid (5.0 g, 32.9 mmol), CuI (0.8 g, 4.2 mmol), dimethylglycine (1.0 g, 9.7 mmol), Cs₂CO₃ (7.8 g, 23.9 mmol) were sequentially added, evacuated, and backfilled with argon. Dioxane (20 mL) was added using a syringe. The reaction mixture was heated to 90° C., until the starting material S2a disappeared, as indicated by TLC. The mixture was partitioned between ethyl acetate and a saturated solution of sodium bicarbonate; the organic layer was washed with brine, dried over Na₂SO₄, and concentrated in vacuum. The residue was purified by column chromatography on silica to provide S2b as solid. (1.8 g, yield of 56%).

In a 250 mL flask with a magnetic stirring bar, S2b (1.5 g, 4.2 mmol), tetrahydrofuran (20 mL), methanol (20 mL), sodium hydroxide solution (2.0 M) (20 mL) was sequentially added. The reaction mixture was stirred at room temperature, until the starting material S2b disappeared, as indicated by TLC. The mixture was diluted by 80 mL water, concentrated in vacuum to remove methanol and tetrahydrofuran. The aqueous was adjusted to pH 1.0 by 1.0 N HCl solution, white solid was precipitated, filtrated and dried under vacuum. In a 250 mL flask with a magnetic stirring bar, the white solid was dissolved in methanol (40 mL). Amberlyst-15 (15 g) was added. The reaction mixture was stirred at room temperature, until the starting material disappeared, as indicated by TLC. Amberlyst-15 was removed by filtration and washed by methanol (20 mL×3). The methanol solution was concentrated in vacuum. The residue was purified by column chromatography on silica to provide S2c as solid. (0.69 g, yield of 54%).

Procedure for the preparation of compound S3a. In a 250 mL flask with a magnetic stirring bar, 3-hydroxybenzoic acid (3.05 g, 22.0 mmol) was dissolved in 60 mL of dimethylsulfoxide. Sodium hydride (1.6 g, 42 mmol) was added in batches for half an hour. Next, methyl 4-fluorobenzoate (3.24 g, 21.0 mmol) was added. The reaction mixture was heated to 70° C., until the starting material disappeared, as indicated by TLC. The mixture was diluted by 140 mL water, then adjusted to pH 1.0 by 1.0 N HCl solution. The white solid was precipitated, filtrated and dried under vacuum, S3a (3.7 g, yield of 65%) was obtained as white solid.

Procedure for the preparation of compound S4c. In a 100 mL flask with a magnetic stirring bar, methyl 3-hydroxybenzoic acid (3.0 g, 19.7 mmol), ethyl 2-(4-fluorophenyl)-2-oxoacetate (4.0 g, 20.4 mmol), K₂CO₃ (5.6 g, 40.5 mmol) and dimethylsulfoxide (60 mL) were sequentially added. The reaction mixture was heated to 70° C., until the starting material disappeared, as indicated by TLC. The mixture was partitioned between ethyl acetate and a saturated solution of sodium bicarbonate; the organic layer was washed with brine, dried over Na₂SO₄, and concentrated in vacuum. The residue was purified by column chromatography on silica to provide S4a as oil. (5.8 g, yield of 90%). S4a (5.7 g) was hydrolyzed following the hydrolysis procedure of the S2b, the diacid intermediate (4.0 g, yield of 80%) was obtained as white solid. In a 250 mL flask with a magnetic stirring bar, the diacid intermediate (3.0 g, 10.5 mmol), ethylene glycol (50 mL) and hydrazine monohydrate (3.28 mL, 52.5 mmol) were sequentially added. The reaction mixture was heated to 60° C. for an hour. Next, sodium hydroxide (4.2 g, 105 mmol) was added to the reaction mixture and heated to 150° C. for five hours. The mixture was cooled to room temperature and diluted by 100 mL water, then adjusted to pH 1.0 by 1.0 N HCl solution. The white solid was precipitated, filtrated and dried under vacuum, S4b (2.6 g, yield of 91%) was obtained as white solid. In a 250 mL flask with a magnetic stirring bar, the white solid S4b (1.3 g, 4.8 mmol) was dissolved in methanol (50 mL). Amberlyst-15 (13 g) was added. The reaction mixture was stirred at room temperature, until the starting material disappeared, as indicated by TLC. Amberlyst-15 was removed by filtration and washed by methanol (20 mL×3). The methanol solution was concentrated in vacuum. The residue was purified by column chromatography on silica to provide S4c as solid. (1.0 g, yield of 73%).

Procedure for the preparation of compound S5c. In a 100 mL flask with a magnetic stirring bar, methyl 3-hydroxybenzoic acid (1.52 g, 10.0 mmol), 4-fluorobenzaldehyde (1.3 g, 10.5 mmol), K₂CO₃ (2.9 g, 21 mmol) and dimethylformamide (10 mL) were sequentially added. The reaction mixture was heated to 80° C., until the starting material disappeared, as indicated by TLC. The mixture was partitioned between ethyl acetate and a saturated solution of sodium bicarbonate; the organic layer was washed with brine, dried over Na₂SO₄, and concentrated in vacuum. The residue was purified by column chromatography on silica to provide S5a as oil. (2.4 g, yield of 92%). In a 100 mL flask with a magnetic stirring bar, S5a (1.3 g, 5.0 mmol), malonic acid (780 mg, 7.5 mmol), piperidine (0.15 mL, 1.5 mmol) and pyridine (25 mL) were sequentially added. The reaction mixture was heated to 110° C., until the starting material disappeared, as indicated by TLC. The mixture was concentrated in vacuum. The residue was purified by column chromatography on silica to provide S5b (1.1 g, yield of 74%) as white solid. In a 100 mL flask with a magnetic stirring, S5b (1.1 g, 3.7 mmol), palladium on carbon (10 wt. %) (0.1 g) and methanol (20 mL) were added. A hydrogen balloon was connected with the reaction mixture through a needle. The reaction mixture was stirred at room temperature until the starting material S5b disappeared, as indicated by TLC. The mixture was filtered through celite and washed with methanol (3×5 mL); the mixture was then concentrated in vacuum. Following the procedure of preparation S2c, the reduced intermediate was hydrolyzed and monomethylated, S5c (0.8 g, total yield of 72%) was obtained as white solid.

General procedure for the preparation of compound S2-S6. In a 20 mL flask with a magnetic stirring bar, S1b (305 mg, 1.0 mmol), S2c (303 mg, 1.0 mmol), dimethylformamide (5 mL), N,N-diisopropylethylamine (0.45 mL, 2.5 mmol) and Hexafluorophosphate Azabenzotriazole Tetramethyl Uronium (460 mg, 1.2 mmol) were sequentially added. The reaction mixture was stirred at room temperature, until the starting material disappeared, as indicated by TLC. The mixture was partitioned between ethyl acetate and a saturated solution of sodium bicarbonate; the organic layer was washed with brine, dried over Na₂SO₄, and concentrated in vacuum. The residue was purified by column chromatography on silica to provide intermediate (492 mg, yield of 83%) as oil. The intermediate (492 mg, 0.83 mmol) was dissolved in mixture solvent (6 mL, 50% (vol/vol) trifluoroacetic acid in dichloromethane) at room temperature for half an hour. The mixture was concentrated in vacuum. The residue was partitioned between ethyl acetate and a saturated solution of sodium carbonate; the organic layer was washed with brine, dried over Na₂SO₄, and concentrated in vacuum. The deprotected intermediate residue was dissolved by 4 mL of methanol and transferred to a 20 mL flask with a magnetic stirring, N-Boc-(methylamino) acetaldehyde (0.16 mL, 0.92 mmol), palladium on carbon (10 wt. %) (0.1 g) were sequentially added. A hydrogen balloon was connected with the reaction mixture through a needle. The reaction mixture was stirred at room temperature until the starting material disappeared, as indicated by TLC. The mixture was filtered through celite and washed with washed with methanol (3×10 mL). The methanol solution was concentrated in vacuum. The residue was purified by column chromatography on silica to provide S2 as oil. (321 mg, total yield of 60%). S3, S4 and S5 were prepared by the same procedure as S2. The deprotected intermediate (116 mg, 0.23 mmol) was dissolved by 1.0 mL of acetonitrile and transferred to a 10 mL vial with a magnetic stirring, 2-bromoethyl methyl ether (22 μL, 0.23 mmol), Cs₂CO₃ (370 mg, 1.1 mmol) were sequentially added. The reaction mixture was stirred at room temperature until the starting material disappeared, as indicated by TLC. The mixture was filtered through celite and washed with washed with ethyl acetate (3×10 mL). The solution was concentrated in vacuum. The residue was purified by column chromatography on silica to provide S6 as oil. (89 mg, yield of 75%).

General procedure for the preparation of linker AL1+3, AL2+3 to AL2+6. In a 100 mL flask with a magnetic stirring bar, tyramine (1.89 g, 13.8 mmol), Di-tert-butyl decarbonate (3.18 g, 14.6 mmol), dichloromethane (20 mL), triethylamine (2 mL, 14.6 mmol) was sequentially added. The reaction mixture was stirred at room temperature, until the starting material disappeared, as indicated by TLC. The mixture was concentrated in vacuum. The residue was dissolved in ethyl acetate (80 mL) and sequentially washed with IN HCl (2×30 mL), water and brine, dried over Na₂SO₄, and concentrated in vacuum. The residue (3.26 g, yield of 100%) was used directly for next step reaction without purification. In a 25 mL flask with a magnetic stirring bar, the Boc protected tyramine (680 mg, 2.86 mmol), K₂CO₃ (0.95 g, 6.87 mmol), acetonitrile (8 mL), methyl 4-bromobutyrate (0.44 mL, 3.44 mmol) were sequentially added. The reaction mixture was heated to 80° C., until the starting material disappeared, as indicated by TLC. The mixture was partitioned between ethyl acetate and a saturated solution of sodium bicarbonate; the organic layer was washed with brine, dried over Na₂SO₄, and concentrated in vacuum. The residue was purified by column chromatography on silica to obtained AL2+3 (736 mg, yield of 76%) as white solid.

General procedure for the preparation of linker RL1 and RL2. In a 250 mL flask with a magnetic stirring bar, N-boc-piperazine (11.0 g, 59.0 mmol), methyl bromoacetate (5.9 mL, 62.0 mmol), acetonitrile (80 mL), triethylamine (16.5 mL, 118 mmol) was sequentially added. The reaction mixture was stirred at room temperature, until the starting material disappeared, as indicated by TLC. The mixture was concentrated in vacuum. The residue was dissolved in ethyl acetate (100 mL) and sequentially washed water, saturated sodium carbonate and brine, dried over Na₂SO₄, and concentrated in vacuum. The residue (14.5 g, yield of 95%) was used directly for next step reaction without purification. The residue (2.3 g, 9.0 mmol) was dissolved in mixture solvent (10 mL, 50% (vol/vol) trifluoroacetic acid in dichloromethane) at room temperature for half an hour. The mixture was concentrated in vacuum. The deprotected intermediate residue was dissolved with 18 mL of methanol and transferred to a 100 mL flask with a magnetic stirring, 1-boc-piperidine-4-carboxaldehyde (2.1 g, 9.9 mmol), palladium on carbon (10 wt. %) (0.2 g), N,N-diisopropylethylamine (4.5 mL, 27.0 mmol) were sequentially added. A hydrogen balloon was connected with the reaction mixture through a needle. The reaction mixture was stirred at room temperature until the starting material disappeared, as indicated by TLC. The mixture was filtered through celite and washed with washed with methanol (3×20 mL). The methanol solution was concentrated in vacuum. The residue was purified by column chromatography on silica to provide RL1 as oil. (2.4 g, total yield of 76%).

General Procedure for the Preparation of 2a-2e, 3a-3e and 3bN (3e was Used as an Example).

In a 20 mL vial with a magnetic stirring bar, RL2 (272 mg, 0.6 mmol), methanol (2 mL), sodium hydroxide solution (2.0 N) (2 mL) was sequentially added. The reaction mixture was stirred at room temperature, until the starting material RL2 disappeared, as indicated by TLC. The mixture was adjusted to pH 7.0 with HCl (2.0 N) (2 mL) and concentrated in high vacuum. Boc-VHL was synthesized based on previously published procedures.⁴⁴ In a 20 mL vial with a magnetic stirring bar, Boc-VHL (327 mg, 0.6 mmol) was dissolved in mixture solvent (6 mL, 50% (vol/vol) trifluoroacetic acid in dichloromethane) and stirred at room temperature until the starting material disappeared, as indicated by TLC. The mixture was concentrated in high vacuum. This Boc deprotected intermediate was dissolved in 3 mL of dimethylformamide and transfer to the vial contained hydrolyzed RL2, a magnetic stirring bar, N,N-diisopropylethylamine (0.43 mL, 2.4 mmol) (Adjust to pH 7.0), (7-Azabenzotriazol-1-yloxy)tripyrrolidinophosphonium hexafluorophosphate (PyAOP) (376 mg, 0.72 mmol) were sequentially added. The reaction mixture was stirred at room temperature, until the starting material disappeared, as indicated by TLC. The mixture was partitioned between ethyl acetate and a saturated solution of sodium carbonate; the organic layer was washed with brine, dried over Na₂SO₄, and concentrated in vacuum. The residue was purified by column chromatography on silica to provide intermediate RL2-VHL (379 mg, yield of 73%). Following the same procedure, S5 (15 mg, 0.023 mmol) was hydrolyzed; intermediate RL2-VHL (20 mg, 0.023 mmol) was deprotected the Boc; then amide coupling by PyAOP (13 mg, 0.025 mmol) afford Boc-3e (20 mg, yield of 63%). In a 20 mL vial with a magnetic stirring bar, Boc-3e (20 mg, 0.0145 mmol) was dissolved in mixture solvent (2 mL, 50% (vol/vol) trifluoroacetic acid in dichloromethane) and stirred at room temperature until the starting material disappeared, as indicated by TLC. The mixture was concentrated in high vacuum. The residue was redissolved in acetonitrile (2 mL), IN HCl methanol solution (44 μL, 0.044 mmol) was added, white solid precipitation was observed. The solvent was removed by high vacuum, 3e (19 mg, 0.0145 mmol) was obtained in x. HCl salts form.

### Compound Characterization Data

tert-butyl 4-(4-((methylamino)methyl)pyridin-2-yl)piperidine-1-carboxylate (S1b) ¹H NMR (400 MHz, CDCl₃) δ 8.45 (d, J=5.0 Hz, 1H), 7.12 (s, 1H), 7.08 (dd, J=5.1, 1.6 Hz, 1H), 3.75 (s, 2H), 2.95-2.71 (m, 4H), 2.46 (s, 3H), 2.0-1.81 (m, 3H), 1.80-1.60 (m, 3H), 1.46 (s, 9H). ¹³C NMR (101 MHz, CDCl₃) δ 164.7, 154.9, 149.5, 149.4, 121.1, 120.3, 79.5, 54.9, 44.7, 44.1, 36.1, 31.8, 28.6.

tert-butyl 2-(4-iodophenoxy)acetate (S2a) ¹H NMR (400 MHz, CDCl₃) δ 7.61-7.52 (m, 2H), 6.72-6.64 (m, 2H), 4.47 (s, 2H), 1.48 (s, 9H). ¹³C NMR (101 MHz, CDCl₃) δ 167.8, 158.0, 138.4, 117.1, 83.9, 82.7, 65.8, 28.2.

methyl 3-(4-(2-(tert-butoxy)-2-oxoethoxy)phenoxy)benzoate (S2b) ¹H NMR (400 MHz, CDCl₃) δ 7.73 (d, J=7.7 Hz, 1H), 7.62-7.56 (m, 1H), 7.36 (dd, J=8.2, 7.7 Hz, 1H), 7.15 (dd, J=8.2, 2.6 Hz, 1H), 7.02-6.94 (m, 2H), 6.93-6.86 (m, 2H), 4.51 (s, 2H), 3.88 (s, 3H), 1.49 (s, 9H). ¹³C NMR (101 MHz, CDCl₃) δ 168.2, 166.7, 158.5, 154.6, 150.6, 131.9, 129.8, 123.9, 122.5, 120.9, 118.6, 116.1, 82.6, 66.4, 52.3, 28.2.

3-(4-(2-methoxy-2-oxoethoxy)phenoxy)benzoic acid (S2c) ¹H NMR (400 MHz, DMSO-d₆) δ 7.64 (d, J=7.7 Hz, 1H), 7.48 (dd, J=8.1, 7.7 Hz, 1H), 7.38-7.30 (m, 1H), 7.23 (dd, J=8.1, 2.7 Hz, 1H), 7.08-7.02 (m, 2H), 7.02-6.95 (m, 2H), 4.80 (s, 2H), 3.71 (s, 3H). ¹³C NMR (101 MHz, DMSO-d₆) δ 169.3, 166.8, 158.2, 154.4, 149.4, 132.5, 130.3, 123.5, 121.9, 121.2, 117.0, 116.1, 65.0, 51.9.

3-(4-(methoxycarbonyl)phenoxy)benzoic acid (S3a) ¹H NMR (400 MHz, CDCl₃) δ 8.08-8.00 (m, 2H), 7.93 (d, J=7.8 Hz, 1H), 7.80-7.75 (m, 1H), 7.49 (dd, J=8.1, 7.8 Hz, 1H), 7.32 (dd, J=8.1, 2.6 Hz, 1H), 7.05-6.98 (m, 2H), 3.91 (s, 3H). ¹³C NMR (101 MHz, CDCl₃) δ 171.4, 166.7, 161.2, 156.2, 132.0, 131.5, 130.4, 126.2, 125.4, 125.3, 121.4, 117.8, 52.2.

methyl 3-(4-(2-ethoxy-2-oxoacetyl)phenoxy)benzoate (S4a) ¹H NMR (400 MHz, CDCl₃) δ 8.05-7.99 (m, 2H), 7.91 (ddd, J=7.8, 2.6, 1.1 Hz, 1H), 7.74 (dd, J=2.5, 1.5 Hz, 1H), 7.49 (dd, J=8.1, 7.8 Hz, 1H), 7.29 (ddd, J=8.1, 2.5, 1.1 Hz, 1H), 7.05-7.01 (m, 2H), 4.43 (q, J=7.2 Hz, 2H), 3.91 (s, 3H), 1.42 (t, J=7.1 Hz, 3H). ¹³C NMR (101 MHz, CDCl₃) δ 184.9, 166.2, 163.9, 163.2, 155.2, 132.8, 132.5, 130.4, 127.6, 126.3, 125.1, 121.5, 117.7, 62.5, 52.5, 14.2.

3-(4-(carboxymethyl)phenoxy)benzoic acid (S4b) ¹H NMR (400 MHz, DMSO-d₆) δ 7.72-7.67 (m, 1H), 7.53-7.46 (m, 1H), 7.45-7.41 (m, 1H), 7.35-7.24 (m, 3H), 7.07-6.96 (m, 2H), 3.58 (s, 2H). ¹³C NMR (101 MHz, DMSO-d₆) δ 172.8, 166.8, 157.4, 154.7, 132.7, 131.3, 131.0, 130.5, 124.1, 122.8, 119.3, 118.1, 40.0.

3-(4-(2-methoxy-2-oxoethyl)phenoxy)benzoic acid (S4c) ¹H NMR (400 MHz, CDCl₃) δ 7.91-7.84 (m, 1H), 7.74 (dd, J=2.6, 1.5 Hz, 1H), 7.45 (dd, J=8.1, 7.9 Hz, 1H), 7.33-7.25 (m, 3H), 7.05-6.97 (m, 2H), 3.74 (s, 3H), 3.65 (s, 2H). ¹³C NMR (101 MHz, CDCl₃) δ 172.2, 171.7, 157.6, 155.8, 131.2, 131.0, 130.0, 129.6, 125.1, 124.3, 120.1, 119.4, 52.3, 40.5.

methyl 3-(4-formylphenoxy)benzoate (S5a) ¹H NMR (400 MHz, CDCl₃) δ 9.93 (s, 1H), 7.98-7.81 (m, 3H), 7.73 (dd, J=2.5, 1.5 Hz, 1H), 7.48 (dd, J=8.1, 7.9 Hz, 1H), 7.28 (ddd, J=8.1, 2.5, 1.1 Hz, 1H), 7.12-7.02 (m, 2H), 3.91 (s, 3H). ¹³C NMR (101 MHz, CDCl₃) δ 190.9, 166.3, 162.7, 155.5, 132.5, 132.2, 131.8, 130.3, 126.1, 125.0, 121.3, 118.0, 52.5.

(E)-3-(4-(3-(methoxycarbonyl)phenoxy)phenyl) acrylic acid (S5b) ¹H NMR (400 MHz, DMSO-d₆) δ 7.79-7.72 (m, 3H), 7.63-7.55 (m, 2H), 7.50 (dd, J=2.6, 1.5 Hz, 1H), 7.38 (ddd, J=8.2, 2.6, 1.1 Hz, 1H), 7.11-7.02 (m, 2H), 6.47 (d, J=16.0 Hz, 1H), 3.83 (s, 3H). ¹³C NMR (101 MHz, DMSO-d₆) δ 167.6, 165.5, 157.8, 156.3, 143.1, 131.6, 130.8, 130.4, 130.0, 124.6, 124.0, 119.0, 118.9, 118.5, 52.4.

3-(4-(3-methoxy-3-oxopropyl)phenoxy)benzoic acid (S5c) ¹H NMR (400 MHz, CDCl₃) δ 7.88-7.82 (m, 1H), 7.75-7.69 (m, 1H), 7.44 (dd, J=8.2, 7.9 Hz, 1H), 7.26 (dd, J=8.2, 2.6 Hz, 1H), 7.24-7.18 (m, 2H), 7.02-6.95 (m, 2H), 3.71 (s, 3H), 2.98 (t, J=7.7 Hz, 2H), 2.67 (t, J=7.7 Hz, 2H). ¹³C NMR (101 MHz, CDCl₃) δ 173.5, 171.7, 157.9, 154.9, 136.3, 131.1, 130.0, 129.9, 124.8, 124.0, 119.7, 119.5, 51.8, 35.9, 30.3.

methyl 2-(4-(3-(((2-(1-(2-((tert-butoxycarbonyl)(methyl)amino)ethyl)piperidin-4-yl)pyridin-4-yl)methyl)(methyl)carbamoyl)phenoxy)phenoxy)acetate (S2) ¹H NMR (400 MHz, CDCl₃) δ 8.48 (d, J=5.1 Hz, 1H), 7.42-7.27 (m, 1H), 7.15-6.77 (m, 9H), 4.76-4.36 (m, 4H), 3.81 (s, 3H), 3.54-3.31 (m, 2H), 3.31-2.83 (m, 8H), 2.82-2.50 (m, 3H), 2.49-1.65 (m, 6H), 1.45 (s, 9H). ¹³C NMR (101 MHz, CDCl₃) δ 171.2, 169.4, 158.6, 155.9, 154.5, 150.6, 149.7, 146.5, 137.3, 130.1, 121.1, 120.6, 119.9, 119.1, 118.5, 116.4, 116.1, 115.4, 79.6, 65.9, 56.2, 54.1, 52.4, 50.3, 46.4, 44.0, 37.6, 35.0, 31.6, 28.6.

methyl 4-(3-(((2-(1-(2-((tert-butoxycarbonyl)(methyl)amino)ethyl)piperidin-4-yl)pyridin-4-yl)methyl)(methyl)carbamoyl)phenoxy)benzoate (S3) ¹H NMR (400 MHz, CDCl₃) δ 8.49 (d, J=5.1 Hz, 1H), 8.07-7.90 (m, 2H), 7.50-7.32 (m, 1H), 7.25-6.81 (m, 7H), 4.76-4.41 (m, 2H), 3.89 (s, 3H), 3.51-3.30 (m, 2H), 3.26-2.99 (m, 3H), 2.96-2.81 (m, 5H), 2.79-2.47 (m, 3H), 2.39-2.11 (m, 2H), 2.06-1.71 (m, 4H), 1.45 (s, 9H). ¹³C NMR (101 MHz, CDCl₃) δ 170.8, 166.5, 161.1, 156.0, 149.7, 146.4, 137.7, 131.9, 130.5, 125.3, 123.0, 122.3, 121.3, 120.6, 119.9, 119.3, 118.6, 117.9, 79.6, 56.3, 54.2, 52.2, 50.4, 46.4, 44.1, 37.6, 34.9, 31.7, 28.5.

methyl 2-(4-(3-(((2-(1-(2-((tert-butoxycarbonyl)(methyl)amino)ethyl)piperidin-4-yl)pyridin-4-yl)methyl)(methyl)carbamoyl)phenoxy)phenyl)acetate (S4) ¹H NMR (400 MHz, CDCl₃) δ 8.49 (d, J=5.2 Hz, 1H), 7.44-7.15 (m, 3H), 7.13-6.70 (m, 7H), 4.77-4.38 (m, 2H), 3.70 (s, 3H), 3.60 (s, 2H), 3.43-3.28 (m, 2H), 3.13-2.85 (m, 8H), 2.76-2.65 (m, 1H), 2.63-2.42 (m, 2H), 2.27-2.05 (m, 2H), 2.02-1.70 (m, 4H), 1.45 (s, 9H). ¹³C NMR (101 MHz, CDCl₃) δ 172.1, 171.1, 157.5, 155.8, 149.7, 146.4, 137.4, 130.9, 130.2, 129.6, 121.8, 121.0, 120.4, 120.0, 119.4, 118.6, 117.3, 116.4, 79.5, 56.4, 54.3, 52.2, 50.4, 46.6, 44.3, 40.5, 37.6, 34.8, 31.9, 28.6.

methyl 3-(4-(3-(((2-(1-(2-((tert-butoxycarbonyl)(methyl)amino)ethyl)piperidin-4-yl)pyridin-4-yl)methyl)(methyl)carbamoyl)phenoxy)phenyl)propanoate (S5) ¹H NMR (400 MHz, CDCl₃) δ 8.48 (d, J=5.0 Hz, 1H), 7.40-7.27 (m, 1H), 7.20-6.85 (m, 9H), 4.79-4.38 (m, 2H), 3.67 (s, 3H), 3.44-3.27 (m, 2H), 3.13-2.98 (m, 3H), 2.97-2.82 (m, 7H), 2.76-2.66 (m, 1H), 2.62 (t, J=7.8 Hz, 2H), 2.58-2.40 (m, 2H), 2.24-2.08 (m, 2H), 2.02-1.69 (m, 4H), 1.45 (s, 9H). ¹³C NMR (101 MHz, CDCl₃) δ 173.3, 171.2, 157.8, 155.8, 149.7, 146.4, 137.4, 136.3, 130.1, 129.8, 121.6, 120.8, 120.4, 119.8, 119.5, 118.6, 117.1, 116.2, 79.5, 56.5, 54.3, 51.8, 50.4, 46.6, 44.3, 37.6, 35.8, 34.8, 31.9, 30.3, 28.6.

methyl 2-(4-(3-(((2-(1-(2-methoxyethyl)piperidin-4-yl)pyridin-4-yl)methyl)(methyl)carbamoyl)phenoxy)phenoxy)acetate (S6) ¹H NMR (400 MHz, CDCl₃) δ 8.47 (d, J=5.1 Hz, 1H), 7.40-7.28 (m, 1H), 7.12-6.83 (m, 9H), 4.62 (s, 4H), 3.81 (s, 3H), 3.53 (t, J=5.7 Hz, 2H), 3.35 (s, 3H), 3.16-3.04 (m, 2H), 3.03-2.86 (m, 3H), 2.79-2.49 (m, 3H), 2.24-2.04 (m, 2H), 2.10-1.70 (m, 4H). ¹³C NMR (101 MHz, CDCl₃) δ 171.1, 169.4, 158.5, 154.5, 150.6, 149.7, 146.4, 137.3, 130.1, 121.1, 120.4, 119.6, 119.1, 118.4, 116.4, 116.1, 115.4, 70.3, 65.9, 59.0, 58.2, 54.4, 52.4, 50.3, 44.2, 37.5, 31.7.

methyl 4-(4-(((tert-butoxycarbonyl)amino)methyl)phenoxy) butanoate (AL1+3) ¹H NMR (400 MHz, CDCl₃) δ 7.24-7.08 (m, 2H), 6.90-6.78 (m, 2H), 4.83 (s, 1H), 4.22 (d, J=5.9 Hz, 2H), 3.97 (t, J=6.1 Hz, 2H), 3.67 (s, 3H), 2.51 (t, J=7.3 Hz, 2H), 2.09 (tt, J=7.3, 6.1 Hz, 2H), 1.44 (s, 9H). ¹³C NMR (101 MHz, CDCl₃) δ 173.7, 158.2, 155.9, 131.3, 128.9, 114.6, 79.4, 66.8, 51.7, 44.2, 30.6, 28.5, 24.7.

methyl 4-(4-(2-((tert-butoxycarbonyl)amino)ethyl)phenoxy) butanoate (AL2+3) ¹H NMR (400 MHz, CDCl₃) δ 7.16-7.00 (m, 2H), 6.88-6.73 (m, 2H), 4.57 (s, 1H), 3.97 (t, J=6.1 Hz, 2H), 3.67 (s, 3H), 3.41-3.16 (m, 2H), 2.71 (t, J=7.1 Hz, 2H), 2.51 (t, J=7.3 Hz, 2H), 2.21-2.01 (m, 2H), 1.42 (s, 9H). ¹³C NMR (101 MHz, CDCl₃) δ 173.7, 157.5, 156.0, 131.2, 129.8, 114.7, 79.2, 66.8, 51.7, 42.0, 35.4, 30.6, 28.5, 24.7.

methyl 5-(4-(2-((tert-butoxycarbonyl)amino)ethyl)phenoxy) pentanoate (AL2+4) ¹H NMR (400 MHz, CDCl₃) δ 7.15-6.98 (m, 2H), 6.89-6.71 (m, 2H), 4.57 (s, 1H), 4.01-3.87 (m, 2H), 3.66 (s, 3H), 3.44-3.16 (m, 2H), 2.71 (t, J=7.1 Hz, 2H), 2.46-2.31 (m, 2H), 1.86-1.74 (m, 4H), 1.42 (s, 9H). ¹³C NMR (101 MHz, CDCl₃) δ 174.0, 157.7, 156.0, 131.1, 129.8, 114.6, 79.2, 67.4, 51.6, 42.1, 35.4, 33.8, 28.8, 28.5, 21.7.

methyl 6-(4-(2-((tert-butoxycarbonyl)amino)ethyl)phenoxy) hexanoate (AL2+5) ¹H NMR (400 MHz, CDCl₃) δ 7.15-7.01 (m, 2H), 6.85-6.74 (m, 2H), 4.59 (s, 1H), 3.91 (t, J=6.4 Hz, 2H), 3.65 (s, 3H), 3.40-3.21 (m, 2H), 2.70 (t, J=7.0 Hz, 2H), 2.33 (t, J=7.5 Hz, 2H), 1.82-1.73 (m, 2H), 1.73-1.63 (m, 2H), 1.53-1.45 (m, 2H), 1.42 (s, 9H). 13C NMR (101 MHz, CDCl₃) δ 174.0, 157.7, 155.9, 130.9, 129.7, 114.6, 79.1, 67.6, 51.5, 42.0, 35.3, 34.0, 29.0, 28.4, 25.7, 24.7.

methyl 7-(4-(2-((tert-butoxycarbonyl)amino)ethyl)phenoxy) heptanoate (AL2+6) ¹H NMR (400 MHz, CDCl₃) δ 7.13-7.00 (m, 2H), 6.89-6.74 (m, 2H), 4.59 (s, 1H), 3.91 (t, J=6.4 Hz, 2H), 3.65 (s, 3H), 3.39-3.17 (m, 2H), 2.71 (t, J=7.1 Hz, 2H), 2.30 (t, J=7.5 Hz, 2H), 1.81-1.71 (m, 2H), 1.69-1.60 (m, 2H), 1.51-1.33 (m, 13H). ¹³C NMR (101 MHz, CDCl₃) δ 174.2, 157.8, 155.9, 130.9, 129.7, 114.6, 79.1, 67.8, 51.5, 42.0, 35.3, 34.0, 29.2, 28.9, 28.5, 25.8, 24.9.

tert-butyl 4-((4-(2-methoxy-2-oxoethyl)piperazin-1-yl)methyl)piperidine-1-carboxylate (RL1) ¹H NMR (400 MHz, CDCl₃) δ 3.71 (s, 3H), 3.20 (s, 2H), 2.73-2.34 (m, 10H), 2.18 (d, J=7.0 Hz, 2H), 1.75-1.66 (m, 2H), 1.66-1.58 (m, 1H), 1.46-1.41 (m, 11H), 1.11-0.99 (m, 2H). ¹³C NMR (101 MHz, CDCl₃) δ 170.8, 155.0, 79.3, 64.4, 59.5, 53.4, 53.1, 51.8, 43.9, 33.6, 30.8, 28.5.

(2S,4R)-1-((S)-3,3-dimethyl-2-(4-(4-((2-(4-(3-(methyl((2-(1-(2-(methylamino)ethyl)piperidin-4-yl)pyridin-4-yl)methyl)carbamoyl)phenoxy)phenoxy)acetamido)methyl)phenoxy) butanamido)butanoyl)-4-hydroxy-N—((S)-1-(4-(4-methylthiazol-5-yl)phenyl)ethyl)pyrrolidine-2-carboxamide (2a) ¹H NMR (400 MHz, MeOD) δ 9.67 (s, 1H), 8.73 (d, J=6.3 Hz, 1H), 8.01-7.73 (m, 2H), 7.58-7.37 (m, 5H), 7.34-6.82 (m, 11H), 5.05-4.95 (m, 3H), 4.69-4.51 (m, 4H), 4.50-4.32 (m, 3H), 4.08-3.81 (m, 5H), 3.74 (dd, J=11.0, 4.0 Hz, 1H), 3.66-3.49 (m, 5H), 3.45-3.36 (m, 2H), 3.14 (s, 3H), 2.83 (s, 3H), 2.57 (s, 3H), 2.53-2.28 (m, 6H), 2.25-2.15 (m, 1H), 2.10-2.00 (m, 2H), 1.99-1.87 (m, 1H), 1.59-1.48 (m, 3H), 1.03 (s, 9H). HRMS (ESI/[M+H]⁺) Calcd for [C₆₄H₇₉N₉O₉S+H]⁺: 1150.5794. found: 1150.5804. HPLC purity: 99.1%.

(2S,4R)-1-((S)-3,3-dimethyl-2-(4-(4-(2-(2-(4-(3-(methyl((2-(1-(2-(methylamino)ethyl)piperidin-4-yl)pyridin-4-yl)methyl)carbamoyl)phenoxy)phenoxy)acetamido)ethyl)phenoxy) butanamido)butanoyl)-4-hydroxy-N—((S)-1-(4-(4-methylthiazol-5-yl)phenyl)ethyl)pyrrolidine-2-carboxamide (2b) ¹H NMR (400 MHz, MeOD) δ 9.81 (s, 1H), 8.74 (d, J=6.1 Hz, 1H), 8.06-7.77 (m, 2H), 7.61-7.37 (m, 5H), 7.35-6.79 (m, 11H), 5.08-4.94 (m, 3H), 4.66-4.31 (m, 5H), 4.05-3.81 (m, 5H), 3.74 (dd, J=11.1, 4.0 Hz, 1H), 3.65-3.36 (m, 9H), 3.14 (s, 3H), 2.83 (s, 3H), 2.77 (t, J=7.4 Hz, 2H), 2.59 (s, 3H), 2.54-2.32 (m, 6H), 2.26-2.15 (m, 1H), 2.08-2.00 (m, 2H), 1.98-1.88 (m, 1H), 1.60-1.46 (m, 3H), 1.03 (s, 9H). HRMS (ESI/[M+H]⁺) Calcd for [C₆₅H₈₁NO₉S+H]⁺: 1164.5951. found: 1164.5938. HPLC purity: 99.6%.

(2S,4R)-1-((S)-3,3-dimethyl-2-(5-(4-(2-(2-(4-(3-(methyl((2-(1-(2-(methylamino)ethyl)piperidin-4-yl)pyridin-4-yl)methyl)carbamoyl)phenoxy)phenoxy)acetamido)ethyl)phenoxy)pentanamido)butanoyl)-4-hydroxy-N—((S)-1-(4-(4-methylthiazol-5-yl)phenyl)ethyl)pyrrolidine-2-carboxamide (2c) ¹H NMR (400 MHz, MeOD) δ 9.50 (s, 1H), 8.73 (d, J=6.1 Hz, 1H), 8.01-7.70 (m, 2H), 7.61-7.36 (m, 5H), 7.35-6.78 (m, 11H), 5.05-4.95 (m, 3H), 4.65-4.33 (m, 5H), 4.13-3.80 (m, 5H), 3.75 (dd, J=11.0, 4.0 Hz, 1H), 3.65-3.36 (m, 9H), 3.13 (s, 3H), 2.83 (s, 3H), 2.78 (t, J=7.0 Hz, 2H), 2.55 (s, 3H), 2.48-2.26 (m, 6H), 2.24-2.14 (m, 1H), 1.99-1.90 (m, 1H), 1.87-1.70 (m, 4H), 1.59-1.47 (m, 3H), 1.04 (s, 9H). HRMS (ESI/[M+H]⁺) Calcd for [C₆₆H₈₃N₉O₉S+H]⁺: 1178.6107. found: 1178.6109. HPLC purity: 99.0%.

(2S,4R)-1-((S)-3,3-dimethyl-2-(6-(4-(2-(2-(4-(3-(methyl((2-(1-(2-(methylamino)ethyl)piperidin-4-yl)pyridin-4-yl)methyl)carbamoyl)phenoxy)phenoxy)acetamido)ethyl)phenoxy) hexanamido)butanoyl)-4-hydroxy-N—((S)-1-(4-(4-methylthiazol-5-yl)phenyl)ethyl)pyrrolidine-2-carboxamide (2d) ¹H NMR (400 MHz, MeOD) δ 9.73 (s, 1H), 8.74 (d, J=6.0 Hz, 1H), 8.07-7.72 (m, 2H), 7.59-7.39 (m, 5H), 7.35-6.78 (m, 11H), 5.07-4.93 (m, 3H), 4.64-4.33 (m, 5H), 4.03-3.80 (m, 5H), 3.74 (dd, J=11.0, 3.9 Hz, 1H), 3.66-3.36 (m, 9H), 3.14 (s, 3H), 2.83 (s, 3H), 2.77 (t, J=7.4 Hz, 2H), 2.58 (s, 3H), 2.49-2.16 (m, 7H), 1.98-1.89 (m, 1H), 1.83-1.61 (m, 4H), 1.60-1.43 (m, 5H), 1.04 (s, 9H). ¹³C NMR (126 MHz, MeOD) δ 175.9, 173.7, 173.3, 172.3, 171.0, 161.4, 160.0, 159.2, 158.3, 155.9, 155.6, 151.8, 147.4, 144.2, 143.1, 137.8, 136.4, 132.1, 131.4, 130.8, 130.6, 128.9, 128.1, 127.8, 125.0, 124.8, 122.5, 122.2, 120.5, 117.3, 115.6, 71.0, 69.1, 68.8, 60.6, 59.1, 58.0, 53.7, 53.6, 52.3, 50.2, 44.3, 41.8, 39.2, 38.8, 37.6, 36.5, 36.4, 35.6, 33.9, 30.1, 29.2, 27.1, 26.8, 26.8, 22.4, 13.8. HRMS (ESI/[M+H]⁺) Calcd for [C₆₇H₈₅N₉O₉S+H]⁺: 1192.6264. found: 1192.6290. HPLC purity: 99.5%.

(2S,4R)-1-((S)-3,3-dimethyl-2-(7-(4-(2-(2-(4-(3-(methyl((2-(1-(2-(methylamino)ethyl)piperidin-4-yl)pyridin-4-yl)methyl)carbamoyl)phenoxy)phenoxy)acetamido)ethyl)phenoxy) heptanamido)butanoyl)-4-hydroxy-N—((S)-1-(4-(4-methylthiazol-5-yl)phenyl)ethyl)pyrrolidine-2-carboxamide (2e) ¹H NMR (400 MHz, MeOD) δ 9.89 (s, 1H), 8.81-8.62 (m, 1H), 8.07-7.73 (m, 2H), 7.65-7.43 (m, 5H), 7.39-6.67 (m, 11H), 5.06-4.96 (m, 3H), 4.65-4.33 (m, 5H), 4.05-3.83 (m, 5H), 3.74 (dd, J=10.9, 3.9 Hz, 1H), 3.64-3.38 (m, 9H), 3.14 (s, 3H), 2.83 (s, 3H), 2.78 (t, J=7.4 Hz, 2H), 2.60 (s, 3H), 2.46-2.16 (m, 7H), 1.98-1.86 (m, 1H), 1.81-1.60 (m, 4H), 1.54-1.36 (m, 7H), 1.04 (s, 9H). HRMS (ESI/[M+H]⁺) Calcd for [C₆₈H₈₇N₉O₉S+H]⁺: 1206.6420. found: 1206.6421. HPLC purity: 99.9%.

(2S,4R)-1-((S)-3,3-dimethyl-2-(2-(4-((1-(2-(4-(3-(methyl((2-(1-(2-(methylamino)ethyl)piperidin-4-yl)pyridin-4-yl)methyl)carbamoyl)phenoxy)phenoxy)acetyl)piperidin-4-yl)methyl)piperazin-1-yl)acetamido)butanoyl)-4-hydroxy-N—((S)-1-(4-(4-methylthiazol-5-yl)phenyl)ethyl)pyrrolidine-2-carboxamide (3a) ¹H NMR (400 MHz, MeOD) δ 9.39 (s, 1H), 8.79-8.66 (m, 1H), 7.98-7.64 (m, 2H), 7.59-7.35 (m, 5H), 7.31-6.66 (m, 7H), 5.04-4.97 (m, 3H), 4.66-4.43 (m, 4H), 4.07-3.71 (m, 6H), 3.71-3.35 (m, 16H), 3.24-3.02 (m, 8H), 2.91-2.73 (m, 4H), 2.53 (s, 3H), 2.50-2.11 (m, 6H), 2.09-1.83 (m, 3H), 1.62-1.49 (m, 3H), 1.47-1.33 (m, 2H), 1.07 (s, 9H). HRMS (ESI/[M+H]⁺) Calcd for [C₆₅H₈₇N₁₁O₈S+H]⁺: 1182.6533. found: 1182.6534. HPLC purity: 99.3%.

(2S,4R)-1-((S)-3,3-dimethyl-2-(2-(4-((1-((1-(2-(4-(3-(methyl((2-(1-(2-(methylamino)ethyl)piperidin-4-yl)pyridin-4-yl)methyl)carbamoyl)phenoxy)phenoxy)acetyl)piperidin-4-yl)methyl)piperidin-4-yl)methyl)piperazin-1-yl)acetamido)butanoyl)-4-hydroxy-N—((S)-1-(4-(4-methylthiazol-5-yl)phenyl)ethyl)pyrrolidine-2-carboxamide (3b) ¹H NMR (400 MHz, MeOD) δ 9.76 (s, 1H), 8.75 (d, J=6.3 Hz, 1H), 8.05-7.70 (m, 2H), 7.62-7.39 (m, 5H), 7.35-6.61 (m, 7H), 5.09-4.95 (m, 3H), 4.67-4.35 (m, 4H), 4.10-3.34 (m, 26H), 3.29-2.95 (m, 10H), 2.84 (s, 4H), 2.59 (s, 3H), 2.50-2.09 (m, 9H), 1.99-1.70 (m, 5H), 1.64-1.49 (m, 3H), 1.44-1.28 (m, 2H), 1.07 (s, 9H). ¹³C NMR (126 MHz, MeOD) δ 173.8, 173.2, 171.6, 168.7, 161.5, 160.1, 158.3, 156.3, 155.8, 147.5, 143.9, 143.1, 137.7, 136.6, 131.4, 130.7, 130.6, 128.7, 128.1, 127.9, 125.1, 124.8, 122.3, 122.1, 120.5, 117.3, 117.2, 71.0, 68.1, 63.4, 62.1, 60.5, 59.4, 58.0, 53.7, 52.3, 51.1, 50.6, 50.2, 45.5, 44.3, 42.6, 39.3, 39.0, 38.8, 36.6, 34.0, 32.4, 31.5, 30.7, 30.3, 29.2, 28.5, 27.0, 26.9, 22.4, 13.6. HRMS (ESI/[M+H]⁺) Calcd for [C₇₁H₉₈N₁₂O₈S+H]⁺: 1279.7424. found: 1279.7440. HPLC purity: 99.4%.

(2S,4R)-4-hydroxy-1-((S)-2-(2-(4-((1-((1-(2-(4-(3-(((2-(1-(2-methoxyethyl)piperidin-4-yl)pyridin-4-yl)methyl)(methyl)carbamoyl)phenoxy)phenoxy)acetyl)piperidin-4-yl)methyl)piperidin-4-yl)methyl)piperazin-1-yl)acetamido)-3,3-dimethylbutanoyl)-N—((S)-1-(4-(4-methylthiazol-5-yl)phenyl)ethyl)pyrrolidine-2-carboxamide (3bN) ¹H NMR (400 MHz, MeOD) δ 9.85 (s, 1H), 8.75 (d, J=5.9 Hz, 1H), 8.12-7.71 (m, 2H), 7.64-7.38 (m, 5H), 7.30-6.64 (m, 7H), 5.10-4.97 (m, 3H), 4.67-4.35 (m, 4H), 4.14-3.34 (m, 29H), 3.30-3.02 (m, 10H), 2.90-2.75 (m, 1H), 2.60 (s, 3H), 2.46-2.16 (m, 9H), 2.02-1.88 (m, 3H), 1.87-1.68 (m, 2H), 1.63-1.49 (m, 3H), 1.45-1.31 (m, 2H), 1.08 (s, 9H). HRMS (ESI/[M+H]⁺) Calcd for [C₇₁H₉₇N₁₁O₉S+H]⁺: 1280.7264. found: 1280.7287. HPLC purity: 99.4%.

(2S,4R)-1-((S)-3,3-dimethyl-2-(2-(4-((1-((1-(4-(3-(methyl((2-(1-(2-(methylamino)ethyl)piperidin-4-yl)pyridin-4-yl)methyl)carbamoyl)phenoxy)benzoyl)piperidin-4-yl)methyl)piperidin-4-yl)methyl)piperazin-1-yl)acetamido)butanoyl)-4-hydroxy-N—((S)-1-(4-(4-methylthiazol-5-yl)phenyl)ethyl)pyrrolidine-2-carboxamide (3c) ¹H NMR (400 MHz, MeOD) δ 9.73-9.46 (m, 1H), 8.75 (d, J=6.1 Hz, 1H), 8.15-7.73 (m, 2H), 7.63-6.89 (m, 12H), 5.08-4.96 (m, 3H), 4.70-4.34 (m, 4H), 3.97-3.81 (m, 5H), 3.79-3.36 (m, 19H), 3.27-2.87 (m, 11H), 2.83 (s, 3H), 2.57 (s, 3H), 2.49-2.11 (m, 9H), 2.00-1.67 (m, 5H), 1.63-1.49 (m, 3H), 1.45-1.31 (m, 2H), 1.07 (s, 9H). HRMS (ESI/[M+H]⁺) Calcd for [C₇₀H₉₆N₁₂O₇S+H]⁺: 1249.7318. found: 1249.7345. HPLC purity: 99.6%.

(2S,4R)-1-((S)-3,3-dimethyl-2-(2-(4-((1-((1-(2-(4-(3-(methyl((2-(1-(2-(methylamino)ethyl)piperidin-4-yl)pyridin-4-yl)methyl)carbamoyl)phenoxy)phenyl)acetyl)piperidin-4-yl)methyl)piperidin-4-yl)methyl)piperazin-1-yl)acetamido)butanoyl)-4-hydroxy-N—((S)-1-(4-(4-methylthiazol-5-yl)phenyl)ethyl)pyrrolidine-2-carboxamide (3d) ¹H NMR (400 MHz, MeOD) δ 9.77 (s, 1H), 8.75 (d, J=6.1 Hz, 1H), 8.13-7.74 (m, 2H), 7.65-7.40 (m, 5H), 7.39-6.78 (m, 7H), 5.11-4.95 (m, 3H), 4.69-4.35 (m, 4H), 4.15-3.86 (m, 6H), 3.85-3.36 (m, 21H), 3.27-3.02 (m, 9H), 2.83 (s, 3H), 2.80-2.67 (m, 1H), 2.59 (s, 3H), 2.52-2.10 (m, 9H), 1.99-1.67 (m, 5H), 1.64-1.49 (m, 3H), 1.26-1.02 (m, 11H). HRMS (ESI/[M+H]⁺) Calcd for [C₇₁H₉₈N₁₂O₇S+H]⁺: 1263.7475. found: 1263.7491. HPLC purity: 99.1%.

(2S,4R)-1-((S)-3,3-dimethyl-2-(2-(4-((1-((1-(3-(4-(3-(methyl((2-(1-(2-(methylamino)ethyl)piperidin-4-yl)pyridin-4-yl)methyl)carbamoyl)phenoxy)phenyl)propanoyl)piperidin-4-yl)methyl)piperidin-4-yl)methyl)piperazin-1-yl)acetamido)butanoyl)-4-hydroxy-N—((S)-1-(4-(4-methylthiazol-5-yl)phenyl)ethyl)pyrrolidine-2-carboxamide (3e) ¹H NMR (400 MHz, MeOD) δ 9.76 (s, 1H), 8.76 (d, J=5.4 Hz, 1H), 8.11-7.77 (m, 2H), 7.67-7.43 (m, 5H), 7.38-6.82 (m, 7H), 5.09-4.97 (m, 3H), 4.70-4.34 (m, 4H), 4.11-3.85 (m, 6H), 3.82-3.36 (m, 19H), 3.28-3.00 (m, 9H), 2.93 (t, J=7.2 Hz, 2H), 2.83 (s, 3H), 2.79-2.64 (m, 3H), 2.59 (s, 3H), 2.52-2.08 (m, 9H), 1.98-1.68 (m, 5H), 1.64-1.49 (m, 3H), 1.26-1.01 (m, 11H). ¹³C NMR (126 MHz, MeOD) δ 173.7, 173.2, 173.0, 171.6, 161.5, 159.5, 156.1, 155.8, 147.5, 143.9, 143.1, 138.3, 137.8, 136.6, 131.4, 131.3, 130.7, 130.6, 128.7, 128.1, 127.9, 125.0, 124.9, 122.8, 121.1, 120.6, 117.9, 71.0, 63.5, 62.1, 60.5, 59.4, 58.0, 53.8, 52.4, 51.0, 50.6, 50.2, 46.4, 44.3, 42.4, 39.3, 39.0, 38.8, 36.6, 35.6, 34.0, 32.3, 31.9, 31.6, 30.8, 30.2, 29.3, 28.5, 27.0, 26.9, 22.4, 13.7. HRMS (ESI/[M+H]⁺) Calcd for [C₇₂H₁₀₀N₁₂O₇S+H]⁺: 1277.7631. found: 1277.7608. HPLC purity: 98.1%.

What is claimed is:

1. A protein degrader, comprising:
a binding molecule configured to bind CARM1;
a linker; and
a ligand configured to bind an E3 ubiquitin ligase;
wherein the binding molecule and the ligand are operationally linked by the linker.

2. The protein degrader of claim 1, wherein the binding molecule is a small molecule.

3. The protein degrader of claim 1, wherein the binding molecule is TP-064:

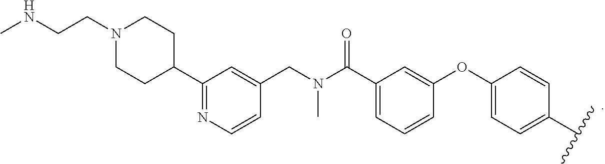 [SMILES: *c1ccc(Oc2cccc(C(=O)N(C)Cc3ccnc(C4CCN(CCNC)CC4)c3)c2)cc1]

4. The protein degrader of claim 1, wherein the ligand is a ligand of Von Hippel-Lindau (VHL) or cereblon (CRBN).

5. The protein degrader of claim 1, wherein the ligand is a VHL ligand.

6. The protein degrader of claim 1, wherein the linker is selected from 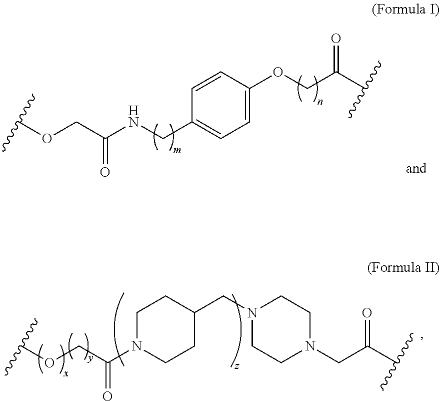
wherein m is 1 or 2; n is an integer from 3 to 7; x is 0 or 1; y is 0, 1, or 2; and z is 1 or 2.

7. The protein degrader of claim 1, wherein the linker is

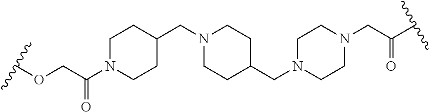 [SMILES: *OCC(=O)N1CCC(CN2CCC(CN3CCN(CC(*)=O)CC3)CC2)CC1]

8. A pharmaceutical composition comprising the protein degrader of claim 1.

9. A method of degrading CARM1 protein, comprising contacting the CARM1 protein with the protein degrader of claim 1.

10. A method comprising administering to an individual in need thereof a therapeutically effective amount of the pharmaceutical composition of claim 8.
